(19) United States
(12) Patent Application Publication
(10) Pub. No.: US 2010/0168038 A1
(43) Pub. Date: Jul. 1, 2010

(54) USE OF COMPOUNDS IN COMBINATION WITH GAMMA-IRRADIATION FOR THE TREATMENT OF CANCER

(76) Inventors: Rickard Glas, Stockholm (SE); Hong Xu, Taby (SE)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/160,787

(22) PCT Filed: Jan. 15, 2007
(86) PCT No.: PCT/EP07/50363

§ 371 Date: Jul. 13, 2008

Related U.S. Application Data
(60) Provisional application No. 60/759,088, filed on Jan. 13, 2006.

(51) Int. Cl. A61K 38/03; A61P 35/00; C12Q 1/34

(52) U.S. Cl. 514/18; 435/18

(57) ABSTRACT

TPP II (tripeptidyl peptidase inhibitors are useful in enhancing the efficacy of gamma-irradiation cancer therapy or increasing the in vivo gamma-irradiation susceptibility of tumour cells. Suitable compounds comprise tripeptide compounds of general formula R^{N1}R^{N2}N-A¹-A²-A³-CO—R^{C1} wherein R^{N1}, R^{N2}, A¹, A², A³ and R^{C1} are as defined herein, and which include for example the tripeptide sequences GLA and GPG. Complete in vivo tumor regression in mice injected with TPPII inhibitors is observed, during treatment in combination with gamma-irradiation.

The present invention relates to the use of compounds in combination with gamma-irradiation for the treatment of cancer.

In the field of cancer therapy, apoptosis resistance is the phenomenon that is usually responsible for irradiation therapy-resistance, i.e. the cancer cells fail to die when encountering gamma-irradiation. Tumours in cancer patients often respond to treatment initially, only to subsequently acquire resistance to therapy. Therapy-resistance of tumour cells is a very common cause for failure of the therapy and death of the patient.

We have now found that gamma-irradiation cancer therapy can be enhanced by using particular compounds. The present invention has arisen from our research into the role of TPP II (tripeptidyl-peptidase II), in DNA damage responses in vitro and in resistance to cancer therapy in vivo. TPP II is built from a unique 138 kDa sub-unit expressed in multi-cellular organisms from Drosophila to Homo Sapiens. Data from Drosophila suggests that the TPP II complex consists of repeated sub-units forming two twisted strands with a native structure of about 6 MDa. TPP II is the only known cytosolic subtilisin-like serine peptidase. Bacterial subtilisins are thoroughly studied enzymes, with numerous reports on crystal structure and enzymatic function (Gupta, R., Beg, Q. K., and Lorenz, P., 2002, “Bacterial alkaline proteases: molecular approaches and industrial applications”, Appl Microbiol Biotechnol. 59:15-32).

Thus, from a first aspect the present invention provides a compound for use in enhancing the efficacy of gamma-irradiation cancer therapy or increasing the in vivo gamma-irradiation susceptibility of tumour cells, wherein said compound is a TPP II inhibitor.

As used herein the term “cancer therapy” covers the treatment of a cancerous condition, as well as preventative therapy and the treatment of a pre-cancerous condition.

As used herein the term “tumour cells” includes cancerous or pre-cancerous cells. Such cells may have cancerous or pre-cancerous defects. Thus the cells may have acquired one or several alterations characteristic of malignant progression.

The invention not only allows gamma-irradiation-resistant tumours to be treated, but is also advantageous even with tumours that can be treated with gamma-irradiation, in allowing lower doses of gamma-irradiation to be used.

From a further aspect the present invention provides a compound for use in enhancing the efficacy of gamma-irradiation cancer therapy or increasing the in vivo gamma-irradiation susceptibility of tumour cells, wherein said compound is selected from the following formula (i) or is a pharmaceutically acceptable salt thereof:

R^{N1}R^{N2}N-A¹-A²-A³-CO—R^{C1} (i)
wherein A¹, A² and A³ are amino acid residues having the following definitions according to the standard one-letter abbreviations or names:
A¹ is G, A, V, L, I, P, 2-aminobutyric acid, norvaline or tert-butyl glycine,
A² is G, A, V, L, I, P, F, W, C, S, K, R, 2-aminobutyric acid, norvaline, norleucine, tert-butyl alanine, alpha-methyl leucine, 4,5-dehydro-leucine, allo-isoleucine, alpha-methyl valine, tert-butyl glycine, 2-allylglycine, ornithine or alpha, gamma-diaminobutyric acid,
A³ is G, A, V, L, I, P, F, W, D, E, Y, 2-aminobutyric acid, norvaline or tert-butyl glycine,
R^{N1} and R^{N2} are each attached to the N terminus of the peptide, are the same or different, and are each independently
   R^{N3},
   (linker1)-R^{N3},
   CO-(linker1)-R^{N3},
   CO—O-(linker1)-R^{N3},
   CO—N-((linker1)-R^{N3})R^{N4} or
   SO₂—(linker1)-R^{N3},
(linker1) may be absent, i.e. a single bond, or CH₂, CH₂CH₂, CH₂CH₂CH₂, CH₂CH₂CH₂CH₂ or CH═CH,
R^{N3} and R^{N4} are the same or different and are hydrogen or any of the following optionally substituted groups:
   saturated or unsaturated, branched or unbranched C₁₋₆ alkyl;
   saturated or unsaturated, branched or unbranched C₃₋₁₂ cycloalkyl;
   benzyl;
   phenyl;
   naphthyl;
   mono- or bicyclic C₁₋₁₀ heteroaryl; or
   non-aromatic C₁₋₁₀ heterocyclyl;
   wherein there may be zero, one or two (same or different) optional substituents on R^{N3} and/or R^{N4} which may be:
      hydroxy-;
      thio-:
      amino-;
      carboxylic acid;
      saturated or unsaturated, branched or unbranched C₁₋₆ alkyloxy;
      saturated or unsaturated, branched or unbranched C₃₋₁₂ cycloalkyl;
      N—, O—, or S— acetyl;
      carboxylic acid saturated or unsaturated, branched or unbranched C₁₋₆ alkyl ester;
      carboxylic acid saturated or unsaturated, branched or unbranched C₃₋₁₂ cycloalkyl ester
      phenyl;
      mono- or bicyclic C₁₋₁₀ heteroaryl;
      non-aromatic C₁₋₁₀ heterocyclyl; or
      halogen;
R^{C1} is attached to the C terminus of the tripeptide, and is:
   O—R^{C2},
   O-(linker2)-R^{C2},
   N((linker2)R^{C2})R^{C3}, or
   N(linker2)R^{C2}—NR^{C3}R^{C4},
(linker2) may be absent, i.e. a single bond, or C₁₋₆ alkyl or C₂₋₄ alkenyl, preferably a single bond or CH₂, CH₂CH₂, CH₂CH₂CH₂, CH₂CH₂CH₂CH₂ or CH═CH,
R^{C2}, R^{C3} and R^{C4} are the same or different, and are hydrogen or any of the following optionally substituted groups:
   saturated or unsaturated, branched or unbranched C₁₋₆ alkyl;
   saturated or unsaturated, branched or unbranched C₃₋₁₂ cycloalkyl;
   benzyl;
   phenyl;
   naphthyl;
   mono- or bicyclic C₁₋₁₀ heteroaryl; or
   non-aromatic C₁₋₁₀ heterocyclyl;
   wherein there may be zero, one or two (same or different) optional substituents on each of R^{C2} and/or R^{C3} and/or R^{C4} which may be one or more of:
      hydroxy-;
      thio-:
      amino-;
      carboxylic acid;
      saturated or unsaturated, branched or unbranched C₁₋₆ alkyloxy;
      saturated or unsaturated, branched or unbranched C₃₋₁₂ cycloalkyl;
      N—, O—, or S— acetyl;
      carboxylic acid saturated or unsaturated, branched or unbranched alkyl ester;
      carboxylic acid saturated or unsaturated, branched or unbranched C₃₋₁₂ cycloalkyl ester
      phenyl;
      halogen;
      mono- or bicyclic C₁₋₁₀ heteroaryl; or
      non-aromatic C₁₋₁₀ heterocyclyl.

The N and CO indicated in the general formula for formula (i) are the nitrogen atom of amino acid residue A¹ and the carbonyl group of amino acid residue A³ respectively.

From a further aspect the invention provides a method of enhancing the efficacy of gamma-irradiation cancer therapy or increasing the in vivo gamma-irradiation susceptibility of tumour cells comprising administering to a patient in need thereof a therapeutically effective amount of a TPPII inhibitor or a compound selected from formula (i) or a pharmaceutically acceptable salt thereof. The compound may be administered in combination with gamma-irradiation cancer therapy in order to decrease resistance to said gamma-irradiation cancer therapy.

The administration of gamma-irradiation, in combination with the compound, is preferably repeated until the tumour is treated, preferably until the tumour disappears.

Similarly, from a further aspect the present invention provides the use of a TPPII inhibitor or a compound selected from formula (i) or a pharmaceutically acceptable salt thereof in the manufacture of a medicament for enhancing the efficacy of gamma-irradiation cancer therapy or increasing the in vivo gamma-irradiation susceptibility of tumour cells.

Without wishing to be bound by theory, the invention may be considered to recognize that TPP II inhibitors are useful in combination with gamma-irradiation in the treatment of cancer.

From a further aspect the present invention provides a pharmaceutical composition comprising a compound of formula (i) or a pharmaceutically acceptable salt thereof and a pharmaceutically acceptable diluent or carrier.

From a further aspect the present invention provides a compound of formula (i) or a pharmaceutically acceptable salt thereof for use as a medicament.

From a further aspect the invention provides a method for identifying a compound suitable for enhancing the efficacy of gamma-irradiation cancer therapy or increasing the in vivo gamma-irradiation susceptibility of tumour cells comprising contacting TPP II with a compound to be screened, and identifying whether the compound inhibits the activity of TPP II.

The present invention recognizes an essential role for TPPII in cellular responses to gamma-irradiation. We have observed complete in vivo tumor regression in mice injected with TPPII inhibitors, during treatment even with relatively low doses of gamma-irradiation.

The present application claims priority from U.S. provisional patent application No. 60/759,088 filed 13 Jan. 2006 by inventors Rickard Glas and Hong Xu and entitled “Use of peptides and peptidomimetic compounds”, the contents of which are hereby incorporated in their entirety, insofar as that application relates to combination with gamma-irradiation for the treatment of cancer. Between the filing of U.S. provisional patent application No. 60/759,099 and the present application, the inventors have carried out further experiments which have enhanced their understanding of the biological mechanisms underlying the present invention. However, the present application is consistent with the earlier priority application in recognizing that TPP II inhibitors are useful in combination with gamma-irradiation for the treatment of cancer, and in identifying particular chemical structures which are preferred in this use.

Without wishing to be bound by theory, our data below indicate that TPPII controls signal transduction by PIKKs, although several points in the mechanism remain to be clarified. TPPII may have a role, direct or indirect, in the recruitment and/or binding of regulatory factors to DNA repair foci, allowing these factors to interact with and become activated by PIKKs. For example, TPPII is believed to control the interaction between ATM and p53 following gamma-irradiation. ATM, ATR and DNA-PKcs have a certain degree of redundancy in stabilization of p53, with multiple N-terminal sites for p53 phosphorylation and with more than one PIKK targeting the same site (Bode, A M, Dong, Z. Post-translational modification of p53 in tumorigenesis. Nat Rev Cancer. 2004; 4:793-805).

TPP II accepts a relatively broad range of substrates. All the compounds falling within formula (i) are peptides or peptide analogues. Compounds of formulae (i) are readily synthesizable by methods known in the art (see for example Ganellin et al., J. Med. Chem. 2000, 43, 664-674) or are readily commercially available (for example from Bachem AG). In a preferred aspect the compound may be selected from formulae (i). Such tripeptides and derivatives are particularly effective therapeutic agents.

According to the invention the compound for use in enhancing the efficacy of gamma-irradiation cancer therapy or increasing the in vivo gamma-irradiation susceptibility of tumour cells may be a compound which is known to be a TPP II inhibitor in vivo.

For example, the compound may be selected from compounds identified in Winter et al., Journal of Molecular Graphics and Modelling 2005, 23, 409-418 as TPP II inhibitors. The compounds may be selected from the following formula (II) because these compounds are particularly suited to the TPP II pharmacophore:

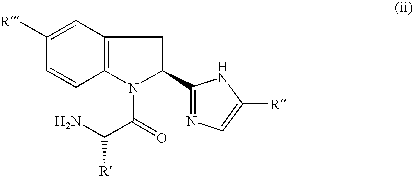

wherein R′ is H, CH₃, CH₂CH₃, CH₂CH₂CH₃ or CH(CH₃)₂,
R″ is H, CH₂CH₃, CH₂CH₂CH₃, CH(CH₃)₂, CH₂CH₂CH₂CH₃CH₂CH(CH₃)₂, CH(CH₃)CH₂CH₃ or C(CH₃)₃, and
R′″ is H, CH₃, OCH₃, F, Cl or Br;

Compounds of formula (II) are synthesizable by known methods (see for example Winter et al., Journal of Molecular Graphics and Modelling 2005, 23, 409-418 and Breslin et al., Bioorg. Med. Chem. Lett. 2003, 13, 4467-4471).

Also by way of example, the compound may be selected from compounds identified in US 6,335,360 of Schwartz et al. as TPP II inhibitors. Such compounds include those of the following formula (iii).

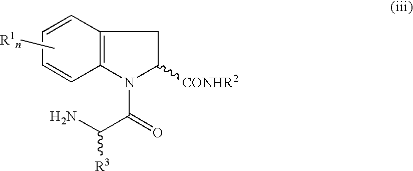

wherein:
each R¹ may be the same or different, and is selected from the group consisting of halogen, OH; C₁-C₆ alkyl optionally substituted by one or more radicals selected from the group consisting of halogen and OH; (C₁-C₆) alkenyl optionally substituted by one or more radicals selected from the group consisting of halogen and OH; (C1-C₆) alkynyl, optionally substituted by one or more radicals selected from the group consisting of halogen and OH, X(C₁-C₆)alkyl, wherein X is S, 0 or OCO, and the alkyl is optionally substituted by one or more radicals selected from the group consisting of halogen and OH; SO₂ (C₁-C₆)alkyl, optionally substituted by at least one halogen, YSO₃H, YSO₂ (C₁-C₆)alkyl, wherein Y is O or NH and the alkyl is optionally substituted by at least one halogen, a diradical —X1-(C₁-C₂)alkylene-X1-wherein X, is O or S; and a benzene ring fused to the indoline ring;
n is from 0 to 4;
R² is CH₂R⁴, wherein R⁴ is C₁-C₆ alkyl substituted by one or more radicals selected from the group consisting of halogen and OH; (CH₂)ₚZ(CH₂)ₚCH₃, wherein Z is O or S, p is from 0 to 5 and q is from 0 to 5, provided that p+q is from 0 to 5; (C₂-C₆) unsaturated alkyl; or (C₃-C₆) cycloalkyl;
or R² is (C₁-C₆)alkyl or O(C₁-C₆)alkyl, each optionally substituted by at least one halogen;
R³ is H; (C₁-C₆)alkyl optionally substituted by at least one halogen; (CH₂)ₚZR⁵ wherein p is from 1 to 3, Z is O or S and R⁵ is H or (C₁-C₃)alkyl; benzyl.

Compounds of formula (iii) are readily synthesizable by known methods (see for example U.S. Pat. No. 6,335,360 of Schwartz et al.).

Nevertheless, it is preferred that the compound be selected from formulae (i) and (ii), more preferably formula (i).

It is also possible for the compound to be a compound of formula (i) wherein R^{N1}, R^{N2} and R^{C1} are as defined above or in any of the preferences below and wherein:
A¹ is G, A, V, L, I, P, S, T, C, N, Q, 2-aminobutyric acid, norvaline, norleucine, tert-butyl alanine, alpha-methyl leucine, 4,5-dehydro-leucine, allo-isoleucine, alpha-methyl valine, tert-butyl glycine or 2-allylglycine,
A² is G, A, V, L, I, P, S, T, C, N, Q, F, Y, W, K, R, histidine, 2-aminobutyric acid, norvaline, norleucine, tert-butyl alanine, alpha-methyl leucine, 4,5-dehydro-leucine, allo-isoleucine, alpha-methyl valine, tert-butyl glycine, 2-allylglycine, ornithine, alpha,gamma-diaminobutyric acid or 4,5-dehydro-lysine, and
A³ is G, A, V, L, I, P, S, T, C, N, Q, D, E, F, Y, W, 2-aminobutyric acid, norvaline, norleucine, tert-butyl alanine, alpha-methyl leucine, 4,5-dehydro-leucine, allo-isoleucine, alpha-methyl valine, tert-butyl glycine or 2-allylglycine.

### Preferred Compounds of Formula (i)

Various groups and specific examples of compounds of formula (i) are preferred.

In general, amino acids of natural (L) configuration are preferred, particularly at the A² position.

In general, it is preferred that R^{N1} is hydrogen, and that
R^{N2} is:
   R^{N3},
   (linked)-R^{N3},
   CO-(linker1)-R^{N3}, or
   CO—O-(linker1)-R^{N3},
wherein
(linker1) may be absent, i.e. a single bond, or CH₂, CH₂CH₂, CH₂CH₂CH₂, CH₂CH₂CH₂CH₂ or CH═CH, and
R^{N3} is hydrogen or any of the following unsubstituted groups:
   saturated or unsaturated, branched or unbranched C₁₋₄ alkyl;
   benzyl;
   phenyl; or
   monocyclic heteroaryl.
In general, it is preferred that R^{C1} is:
   O—R^{C2},
   O-(linker2)-R^{C2}, or
   NH-(linker2)R^{C2}
wherein
(linker2) may be absent, i.e. a single bond, C₁₋₆ alkyl or C₂₋₄ alkenyl, preferably a single bond or CH₂, CH₂CH₂, CH₂CH₂CH₂, CH₂CH₂CH₂CH₂ or CH═CH,
R^{C2} is hydrogen or any of the following unsubstituted groups:
   saturated or unsaturated, branched or unbranched C₁₋₅ alkyl;
   benzyl;
   phenyl; or
   monocyclic C₁₋₁₀ heteroaryl.

In general, with regard to the substituents at the N-terminus, it is further preferred that:

R^{N1} is hydrogen, and
R^{N2} is hydrogen, C(═O)—O-(linker1)-R^{N3} or C(═O)-(linker1)-R^{N3},
(linker1) is CH₂ or CH═CH, and
R^{N3} is phenyl or 2-furyl.

It is further preferred that

R^{N1} is hydrogen,
R^{N2} is hydrogen, C(═O)—OCH₂Ph or C(═O)—CH═CH-(2-furyl).

Another preferred grouping for the substituents on the N-terminus is such that:

R^{N1} is hydrogen, and
R^{N2} is a is benzyloxycarbonyl, benzyl, benzoyl, tert-butyloxycarbonyl, 9-fluorenylmethoxycarbonyl or FA, more preferably benzyloxycarbonyl or FA.

In general, with regard to the substituents at the C-terminus, it is preferred that:

R^{C1} is OH, O—C₁₋₆ alkyl, O—C₁₋₆ alkyl-phenyl, NH—C₁₋₆ alkyl, or NH—C₁₋₆ alkyl-phenyl, more preferably OH.

Several preferred groups are as follows.

Group (i)(a):
A¹ is G, A, V, L, I, P, 2-aminobutyric acid, norvaline or tert-butyl glycine,
A² is G, A, V, L, I, P, F, W, C, S, K, R, 2-aminobutyric acid, norvaline, norleucine, tert-butyl alanine, alpha-methyl leucine, 4,5-dehydro-leucine, allo-isoleucine, alpha-methyl valine, tert-butyl glycine, 2-allylglycine, ornithine or alpha, gamma-diaminobutyric acid,
A³ is G, A, V, L, I, P, F, W, D, E, Y, 2-aminobutyric acid, norvaline or tert-butyl glycine,

### R^{N1} is H,

R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.
Group (i)(b):
A¹ is G, A or 2-aminobutyric acid,
A² is L, I, norleucine, V, norvaline, tert-butyl alanine, 4,5-dehydro-leucine, allo-isoleucine, 2-allylglycine, P, 2-aminobutyric acid, alpha-methyl leucine, alpha-methyl valine or tert-butyl glycine,
A³ is G, A, V, P, 2-aminobutyric acid or norvaline,

### R^{N1} is H,

R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.
Group (i)(c):
A¹ is G, A or 2-aminobutyric acid,
A² is L, I, norleucine, V, norvaline, tert-butyl alanine, 4,5-dehydro-leucine, allo-isoleucine or 2-allylglycine,
A³ is G, A, V, P, 2-aminobutyric acid or norvaline,

### R^{N1} is H,

R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.
Group (i)(d):

### A¹ is G or A,

A² is L, I, or norleucine,

### A³ is G or A,

### R^{N1} is H,

R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

A first set of specific preferred compounds are those in which:

### A¹ is G,

### A² is L,

A³ is G, A, V, L, I, P, F, W, D, E, Y, 2-aminobutyric acid, norvaline or tert-butyl glycine, more preferably G, A, V, P, 2-aminobutyric acid or norvaline, more preferably G or A,
R^{N1} is hydrogen,
R^{N2} is benzyloxycarbonyl, and

### R^{C1} is OH.

A second set of specific preferred compounds are those in which:

### A¹ is G,

A² is G, A, V, L, I, P, F, W, C, S, 2-aminobutyric acid, norvaline, norleucine, tert-butyl alanine, alpha-methyl leucine, 4,5-dehydro-leucine, allo-isoleucine, alpha-methyl valine, tert-butyl glycine or 2-allylglycine, more preferably L, I, norleucine, V, norvaline, tert-butyl alanine, 4,5-dehydro-leucine, allo-isoleucine, 2-allylglycine, P, 2-aminobutyric acid, alpha-methyl leucine, alpha-methyl valine or tert-butyl glycine, more preferably L, I, norleucine, V, norvaline, tert-butyl alanine, 4,5-dehydro-leucine, allo-isoleucine or 2-allylglycine, more preferably L, I, or norleucine,

### A³ is A,

R^{N1} is hydrogen,
R^{N2} is benzyloxycarbonyl, and

### R^{C1} is OH.

A third set of specific preferred compounds are those in which:

A¹ is G, A, V, L, I, P, 2-aminobutyric acid, norvaline or tert-butyl glycine, more preferably G, A or 2-aminobutyric acid, more preferably G or A,

### A² is L,

### A³ is A,

R^{N1} is hydrogen,
R^{N2} is benzyloxycarbonyl, and

### R^{C1} is OH.

Preferably the sequence A¹-A²-A³ is GLA, GLF, GVA, GIA, GPA or ALA, most preferably GLA, and:

R^{N1} is hydrogen,
R^{N2} is benzyloxycarbonyl, and

### R^{C1} is OH.

Where alkyl groups are described as saturated or unsaturated, this encompasses alkyl, alkenyl and alkynyl hydrocarbon moieties.

C₁₋₆ alkyl is preferably C₁₋₄ alkyl, more preferably methyl, ethyl, n-propyl, isopropyl, or butyl (branched or unbranched), most preferably methyl.

C₃₋₁₂ cycloalkyl is preferably C₅₋₁₀ cycloalkyl, more preferably C₅₋₇ cycloalkyl.

“aryl” is an aromatic group, preferably phenyl or naphthyl,
“hetero” as part of a word means containing one or more heteroatom(s) preferably selected from N, O and S.
“heteroaryl” is preferably pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, pyranyl, carbazolyl, acridinyl, quinolinyl, benzimidazolyl, benzthiazolyl, purinyl, cinnolinyl or pteridinyl.
“non-aromatic heterocyclyl” is preferably pyrrolidinyl, piperidyl, piperazinyl, morpholinyl, tetrahydrofuranyl or monosaccharide.
“halogen” is preferably Cl or F, more preferably Cl.

### Further Preferred Compounds of Formula (i)

In general, A¹ may preferably be selected from G, A or 2-aminobutyric acid; more preferably G or A.

In general, A² may preferably be selected from L, I, norleucine, V, norvaline, tert-butyl alanine, 4,5-dehydro-leucine, allo-isoleucine, 2-allylglycine, P, K, 2-aminobutyric acid, alpha-methyl leucine, alpha-methyl valine or tert-butyl glycine; more preferably L, I, norleucine, V, norvaline, tert-butyl alanine, 4,5-dehydro-leucine, allo-isoleucine, 2-allylglycine, P or K; more preferably L, I, norleucine, P or K; more preferably L or P.

In general, A³ may preferably be selected from G, A, V, P, 2-aminobutyric acid or norvaline; more preferably G or A.

In general, it is preferred that R^{N1} is hydrogen.

In general, R^{N2} is preferably:
R^{N3},
(linker1)-R^{N3},
CO-(linker1)-R^{N3}, or
CO—O-(linker1)-R^{N3},
wherein
(linker1) may be absent, i.e. a single bond, or CH₂, CH₂CH₂, CH₂CH₂CH₂, CH₂CH₂CH₂CH₂ or CH═CH, and
R^{N3} is hydrogen or any of the following unsubstituted groups:
   saturated or unsaturated, branched or unbranched C₁₋₄ alkyl;
   benzyl;
   phenyl; or
   monocyclic heteroaryl.

In general, R^{N2} is more preferably hydrogen, benzyloxycarbonyl, benzyl, benzoyl, tert-butyloxycarbonyl, 9-fluorenylmethoxycarbonyl or FA, more preferably hydrogen, benzyloxycarbonyl or FA.

In general, it is preferred that R^{C1} is:
O—R^{C2},
O-(linker1)-R^{C2}, or
NH-(linker2)R^{C2}
wherein
(linker2) may be absent, i.e. a single bond, C₁₋₆ alkyl or C₂₋₄ alkenyl, preferably a single bond or CH₂, CH₂CH₂, CH₂CH₂CH₂, CH₂CH₂CH₂CH₂ or CH═CH,
R^{C2} is hydrogen or any of the following unsubstituted groups:
   saturated or unsaturated, branched or unbranched C₁₋₅ alkyl;
   benzyl;
   phenyl; or
   monocyclic C₁₋₁₀ heteroaryl.

In general, R^{C1} is more preferably OH, O—C₁₋₆ alkyl, O—C₁₋₆ alkyl-phenyl, NH₂, NH—C₁₋₆ alkyl, or NH—C₁₋₆ alkyl-phenyl, more preferably OH, O—C₁₋₆ alkyl, NH₂, or NH—C₁₋₆ alkyl, more preferably OH or NH₂.

Compounds of particular interest include those wherein A² is P.

Compounds of particular interest include those wherein R^{C1} is NH₂.

In general the following amino acids are less preferred for A³: F, W, D, E and Y. Similarly, in general A³ may be selected not to be P and/or E due to compounds containing these exhibiting lower activity.

### Preferred Compounds of Formula (ii)

Compounds of formula (ii) are preferably such that:

### R′ is CH₂CH₃ or CH₂CH₂CH₃,

### R″ is CH₂CH₂CH₃ or CH(CH₃)₂, and

### R′″ is H or Cl.

Preferred Compounds of Formula (iii)

Various preferred groups and specific examples of compounds of formula (iii) are as defined in any of the claims, taken separately, of U.S. Pat. No. 6,335,360 B1 of Schwartz et al.

One example of a therapeutic compound of formula (i) is Z-GLA-OH, i.e. tripeptide GLA which is derivatized at the N-terminus with a Z group and which is not derivatized at the C-terminus. Z denotes benzyloxycarbonyl. This is a compound of formula (i) wherein R^{N1} is H, R^{N2} is Z, A¹ is G, A² is L, A³ is A and R^{C1} is OH. This compound is available commercially from Bachem AG and has been found to inhibit the bacterial homologue of the eukaryotic TPP II, Subtilisin. Z-GLA-OH is of low cost and works well in vivo to induce rejection of tumours that are resistant to therapy with gamma-irradiation. Novel treatments of therapy resistant cancers are of substantial interest to public health.

Whilst preferred compounds include those containing GLA, such as Z-GLA-OH, Bn-GLA-OH, FA-GLA-OH and H-GLA-OH, for example Z-GLA-OH; according to the present invention any disclosures of any compounds or groups of compounds herein may optionally be subject to the proviso that the sequence A¹A²A³ is not GLA, or the proviso that the compound is not selected from the group consisting of Z-GLA-OH, Bn-GLA-OH, FA-GLA-OH or H-GLA-OH, or the proviso that the compound is not Z-GLA-OH.

In the treatment of tumours that fail to respond to standard gamma-irradiation treatment Z-GLA-OH or other compounds described herein may be administered to improve such treatment in patients with malignant disease, for example increasing the in vivo response to such treatment in solid tumours.

Other preferred compounds include those wherein A¹A²A³ is GPG, such as GPG-NH₂ or Z-GPG-NH₂.

The skilled person will be aware that the compounds described herein may be administered in any suitable manner. For example, the administration may be parenteral, such as intravenous or subcutaneous, oral, transdermal, intranasal, by inhalation, or rectal. In one preferred embodiment the compounds are administered by injection.

Examples of pharmaceutically acceptable addition salts for use in the pharmaceutical compositions of the present invention include those derived from mineral acids, such as hydrochlorid, hydrobromic, phosphoric, metaphosphoric, nitric and sulphuric acids, and organic acids, such as tartaric, acetic, citric, malic, lactic, fumaric, benzoic, glycolic, gluconic, succinic, and arylsulphonic acids. The pharmaceutically acceptable excipients described herein, for example, vehicles, adjuvants, carriers or diluents, are well-known to those who are skilled in the art and are readily available to the public. The pharmaceutically acceptable carrier may be one that is chemically inert to the active compounds and that has no detrimental side effects or toxicity under the conditions of use. Pharmaceutical formulations are found e.g. in Remington: The Science and Practice of Pharmacy, 19th ed., Mack Printing Company, Easton, Pa. (1995).

The composition may be prepared for any route of administration, e.g. oral, intravenous, cutaneous or subcutaneous, nasal, intramuscular, or intraperitoneal. The precise nature of the carrier or other material will depend on the route of administration. For a parenteral administration, a parenterally acceptable aqueous solution is employed, which is pyrogen free and has requisite pH, isotonicity and stability. Those skilled in the art are well able to prepare suitable solutions and numerous methods are described in the literature. A brief review of methods of drug delivery is also found in e.g. Langer, Science 249:1527-1533 (1990).

The dose administered to a mammal, particularly a human, in the context of the present invention should be sufficient to effect a therapeutic response in the mammal over a reasonable time frame. One skilled in the art will recognize that dosage will depend upon a variety of factors including the age, condition and body weight of the patient, as well as the stage/severity of the disease. The dose will also be determined by the route (administration form) timing and frequency of administration. In the case of oral administration the dosage can vary for example from about 0.01 mg to about 10 g, preferably from about 0.01 mg to about 1000 mg, more preferably from about 10 mg to about 1000 mg per day of a compound or the corresponding amount of a pharmaceutically acceptable salt thereof.

The compounds may be administered before, during or after gamma-irradiation.

It is clear to the skilled person how to screen compounds for their inhibition of the activity of TPP II. TPP II protein may be purified in a first step, and a TPP II-preferred fluorogenic substrate may be used in a second step. This results in an effective method to measure TPP II activity.

It is not necessary to achieve a particularly high level of purification, and conventional simple techniques can be used to obtain TPP II of sufficient quality to use in a screening method. In one non-limiting example of purification of TPP II, 100×10⁶ cells (such as EL-4 cells) were sedimented and lysed by vortexing in glass beads and homogenisation buffer (50 mM Tris Base pH 7.5, 250 mM Sucrose, 5 mM MgCl₂, 1 mM DTT). Cellular lysates were subjected to differential centrifugation; first the cellular homogenate was centrifuged at 14,000 rpm for 15 min, and then the supernatant was transferred to ultra-centrifugation tubes. Next the sample was ultra-centrifugated at 100,000×g for 1 hour, and the supernatant (denoted as cytosol in most biochemical literature) was subjected to 100,000×g centrifugation for 5 hours, which sedimented high molecular weight cytosolic proteins/protein complexes. The resulting pellet dissolved in 50 mM Tris Base pH 7.5, 30% Glycerol, 5 mM MgCl₂, and 1 mM DTT, and 1 ug of high molecular weight protein was used as enzyme in peptidase assays.

It is possible to test the activity of TPP II using for example the substrate AAF-AMC (Sigma, St. Louis, Mo.). This may for example be used at 100 uM concentration in 100 ul of test buffer composed of 50 mM Tri Base pH 7.5, 5 mM MgCl₂ and 1 mM DTT. It is possible to stop reactions using dilution with 900 ul 1% SDS solution. Cleavage activity may be measured for example by emission at 460 nm in a LS50B Luminescence Spectrometer (Perkin Elmer, Boston, Mass.).

The compounds of use in the present invention may be defined as those which result in partial or preferably complete tumour regression compared to control experiments when used in an in vivo model which comprises the steps of: (i) inoculation of tumor cells into mice; (ii) gamma-irradiation of said mice and administration of compound to said mice; and (iii) measuring the tumour size at periodic intervals. The gamma-irradiation step is omitted in the control experiments. Further details and examples of tumour growth experiments are described below. We found it convenient to inject the compound shortly after application of gamma-irradiation treatment, but the invention should not be understood as limited to this sequence of administration.

The compounds used in the present invention result in partial or preferably complete tumour regression in vivo when applied in combination with gamma-irradiation, for example in a method as described herein.

The compounds used in the present invention are sufficiently serum-stable, i.e. in vivo they retain their identity long enough to exert the desired therapeutic effect.

Without wishing to be bound by theory, the present invention is described in more detail in the non-limiting Examples below with reference to the accompanying drawings which are now summarised.

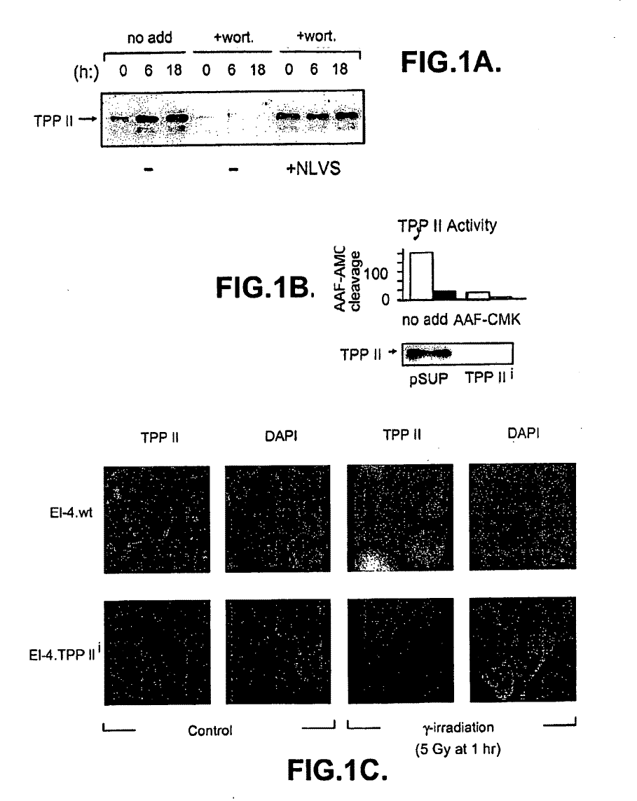

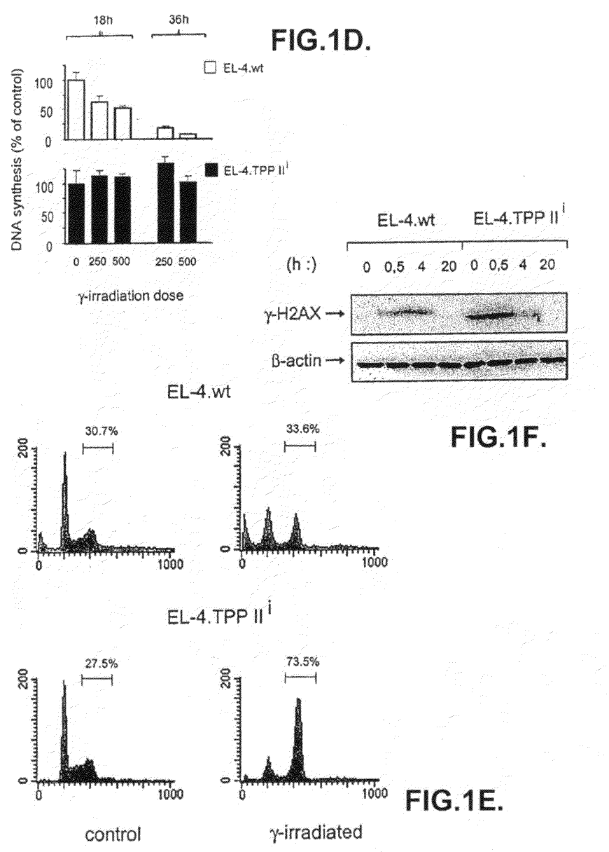

FIG. 1. TPPII in growth arrest regulation by gamma-irradiation exposure. (A) Western blotting analysis using anti-TPPII of cellular lysates from EL-4 cells exposed to 1000 Rad of gamma-irradiation in the presence or absence of 1 micro-M wortmannin; with subsequent exposure to wortmannin in the presence of 25 micro-M NLVS (right lanes). (B) TPPII activity (enzymatic cleavage of AAF-AMC, top) and expression (by western blotting with anti-TPPII, bottom) as determined by testing high molecular weight cytosolic protein from EL-4 cells stably transfected with either empty pSUPER vector (denoted EL-4.wt, empty bars) or with pSUPER-TPPIIi(anti-TPPII siRNA, denoted EL-4.TPPIIⁱ, filled bars). AAF-CMK is a Serine peptidase inhibitor. (C) Immuno-cytochemical analysis of TPPII in EL-4.wt (top) versus EL-4.TPPIIⁱ cells (bottom), either left untreated (left panels) or gamma-irradiated (5 Gy) and analyzed after 1 hour. DAPI was used as controls for nuclear staining. (D) DNA synthesis of gamma-irradiated EL-4.wt (open symbols) and EL-4.TPPIIⁱ cells (closed symbols) following exposure to 1000 Rad, as measured by ³H-Thymidin incorporation (bars indicate +/− standard deviation). (E) Cell cycle analysis of EL-4.wt (top) versus EL-4.TPPIIⁱ cells (bottom), before or 20 hours after exposure to 10 Gy of gamma-irradiation. (F) Phospho-Ser139-H2AX (gamma-H2AX) expression in EL-4.wt control versus EL-4.TPPIIⁱ cells exposed to 2.5 Gy of gamma-irradiation.

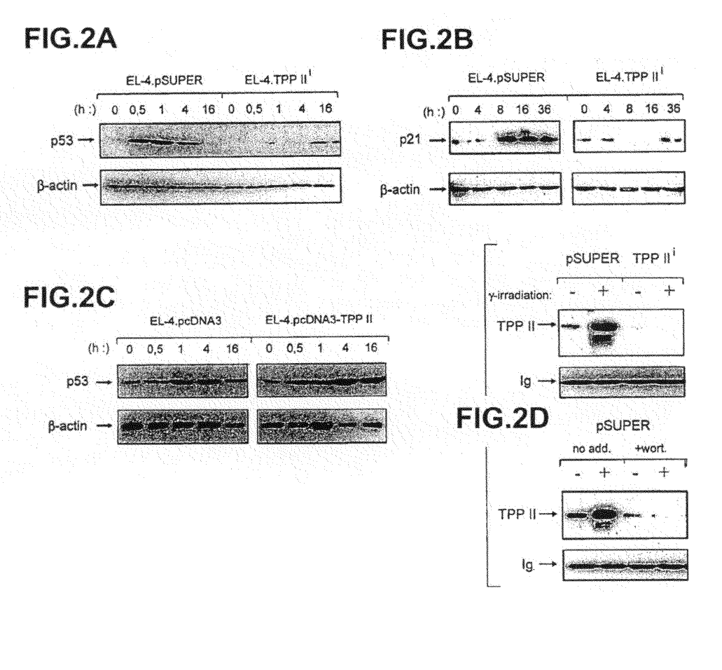

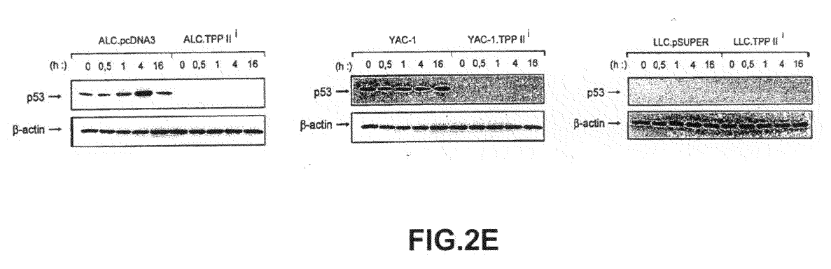

FIG. 2. TPPII expression is required for stabilization of p53. Western blot analysis of cellular lysates after exposure of the indicated cells to gamma-irradiation (10 Gy): (A) p53 expression in EL-4.wt control versus EL-4.TPPIIⁱ cells. (B) p21 expression in EL-4.wt control versus EL-4.TPPIIⁱ cells. (C) p53 expression in EL-4.pcDNA3control versus EL-4.pcDNA3-TPPII cells. (D) Western blotting analysis of TPPII using p53-immunoprecipitates from lysates of EL-4.wt versus EL-4.TPPIIⁱ cells (top); or from EL-4.wt cells treated with 1 micro-M wortmannin, versus untreated (bottom). Lanes labeled “+” indicates gamma-irradiated cells, whereas “−” were untreated (incubated for 16 hours at 37° C., prior to lysis). (E) p53 expression in ALC.pcDNA3 versus ALC.pSUPER-TPPIIⁱ (left), YAC-1 versus YAC-1.pSUPER-TPPIIⁱ (middle) and LLC.pSUPER control versus LLC.TPPIIⁱ cells (right), exposed to gamma-irradiation.

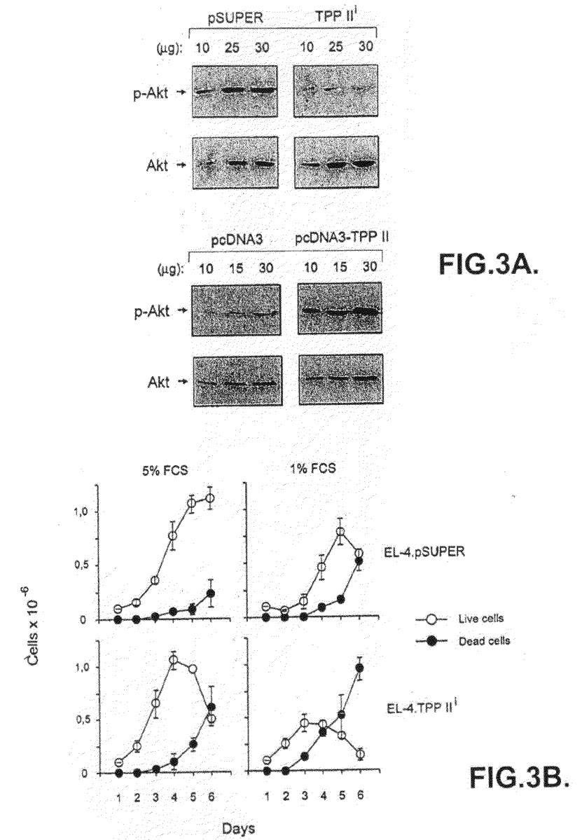

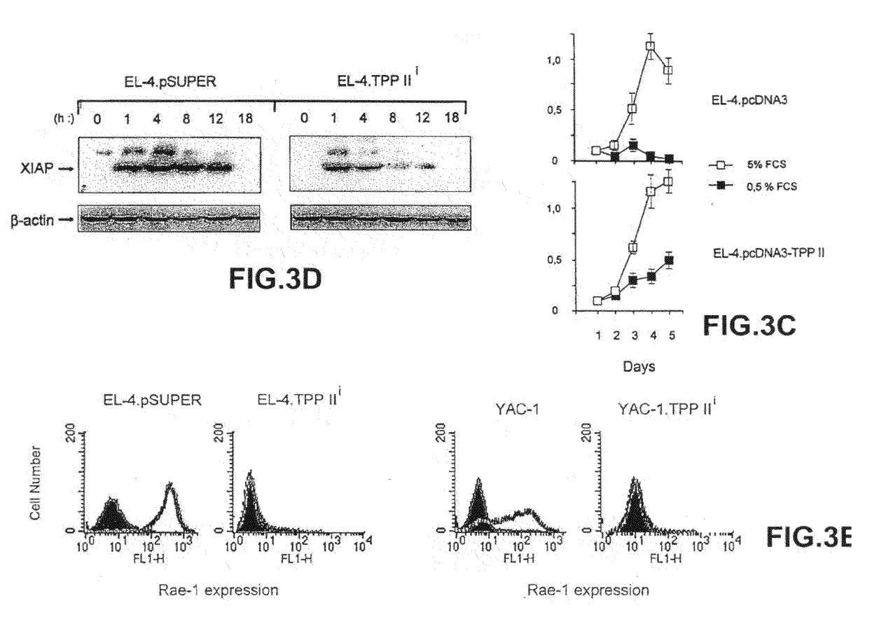

FIG. 3. TPPII controls pathways that respond to PIKK signaling. (A) Western blotting analysis of Akt kinase expression, total Akt and Ser473-phosphorylated (p-Akt), in EL-4.wt control versus EL-4.TPPIIⁱ cells (top), or in EL-4.pcDNA3 versus EL-4.pcDNA3-TPPII cells (bottom). (B) Growth in vitro of EL-4.wt and EL-4.TPPIIⁱ cells in cell culture medium with either high (5%, left) or low (1%, right) serum content. Both live (empty circles) and dead (filled circles) cells were counted. (C) In vitro growth of EL-4.pcDNA3 and EL-4.pcDNA3-TPPII cells in cell culture medium with either high (5%, empty circles) or low (0.5%, filled circles) serum content. (D) XIAP expression by Western blotting analysis in EL-4.wt control cells versus EL-4.TPPIIⁱ cells exposed to 25 micro-M etoposide. (E) Cell surface Rae-1 expression of EL-4 (left) and YAC-1 (right) lymphoma cells with (right panels) and without (left panels) expression of pSUPER-TPPIIⁱ plasmid, as analyzed by flow cytometry. Filled curves represent cells stained with conjugate only.

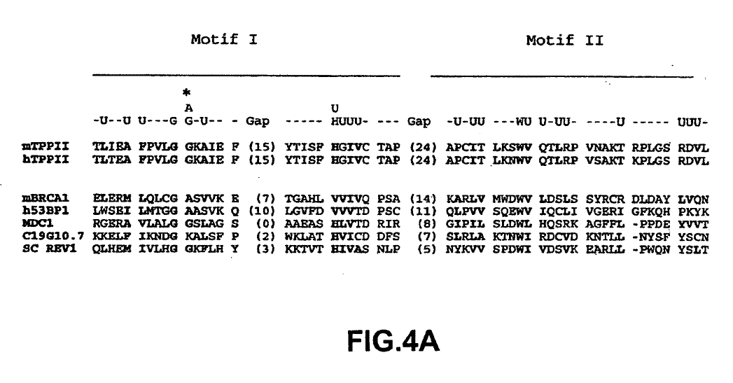

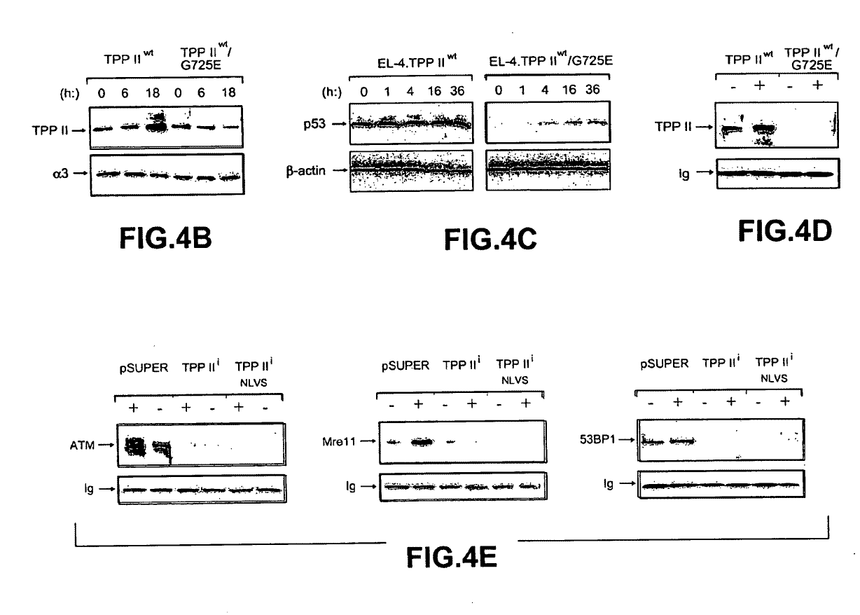

FIG. 4. TPPII controls interactions that mediate p53 stabilization.

(A) Sequence alignment of mouse versus human TPPII (a.a. 715-813) with BRCA C-terminal repeat domains previously described in BRCA1 (mouse), 53BP1 (human), MDC1 (human), C19G10.7 (S. pombe) and Rev1 (S. cerevisiae). U denotes hydrophobic amino acid (Bork, P, Hofmann, K, Bucher, P, Neuwald, A F, Altschul, S F, Koonin, E V. A superfamily of conserved domains in DNA damage-responsive cell cycle checkpoint proteins. FASEB J. 1997; 11:68-76). (B) Western blotting analysis of TPPII using cellular lysates from EL-4.TPPII^{wt} or EL-4.TPPII^{wt}/G725E cells, exposed to 1000 Rad gamma-irradiation or left untreated. (C) p53 expression in EL-4.TPPII^{wt} versus EL-4.TPPII^{wt}/G725E cells exposed to 1000 Rads of gamma-irradiation. (D) Western blotting analysis of TPPII using p53-immuno-precipitates from EL-4.TPPII^{wt} or EL-4.TPPII^{wt}/G725E cells exposed to 1000 Rad gamma-irradiation, or left untreated. (E) Western blotting analysis of p53-immunoprecipitates from lysates of EL-4.wt versus EL-4.TPPIIⁱ cells, treated with NLVS versus untreated, using anti-sera specific for (left) ATM, (middle) Mre11, (right) 53BP1. Lanes labeled “+” indicates gamma-irradiated cells, whereas “−” were untreated.

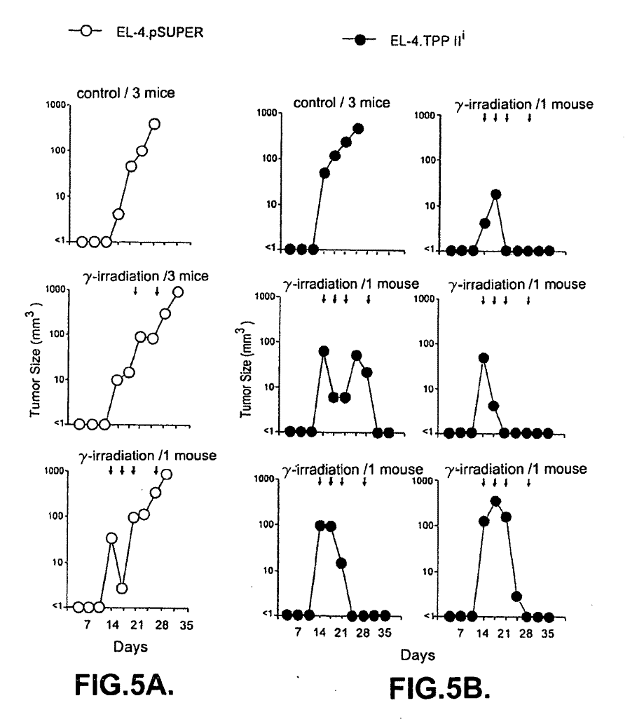

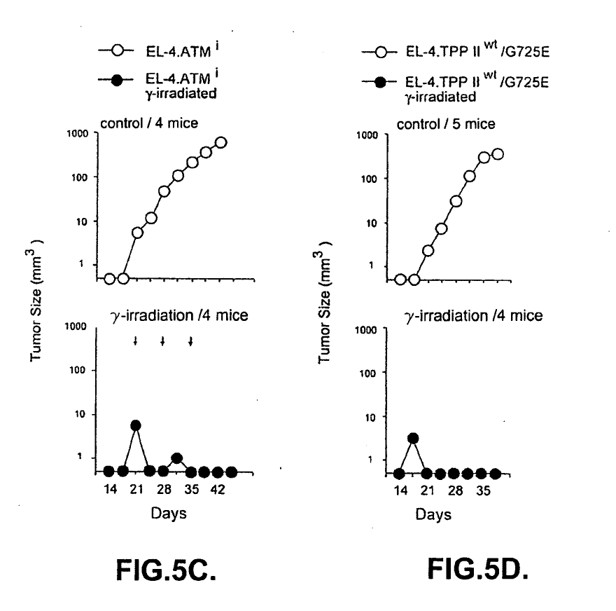

FIG. 5. TPPII is required for in vivo tumor resistance to gamma-irradiation. (A, B) Tumor growth of 10⁶ EL-4.wt (A) or EL-4.TPPIIⁱ cells (B) in syngeneic C57Bl/6 mice, gamma-irradiated with 4 Gy at time-points indicated with arrows. (C) Tumor growth of 5×10⁶ EL-4.ATMⁱ cells in syngeneic C57Bl/6 mice, left untreated (top) or gamma-irradiated with 4 Gy at time-points indicated with arrows (bottom). (D) Tumor growth of 5×10⁶ EL-4.TPPII^{wt}/G725E cells in syngeneic C57Bl/6 mice, left untreated (top) or gamma-irradiated (bottom).

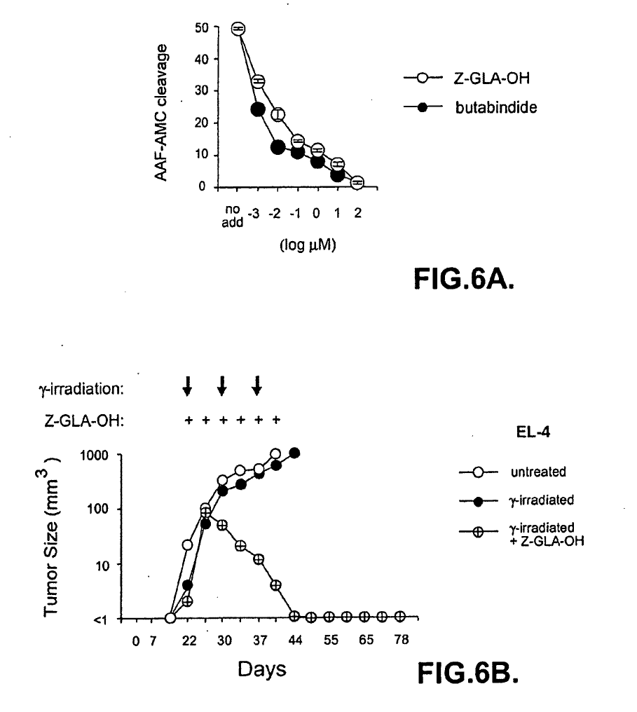

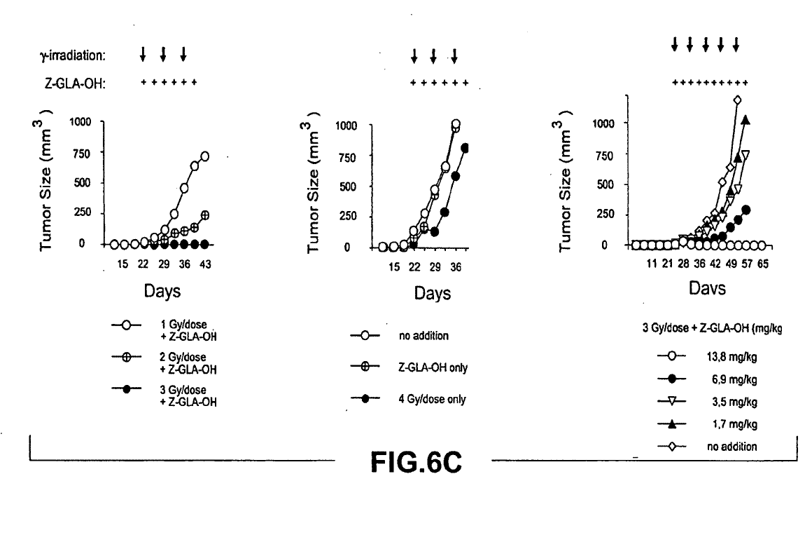

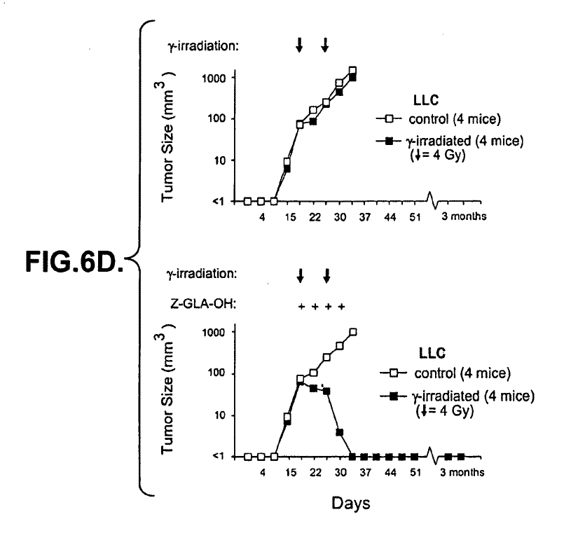

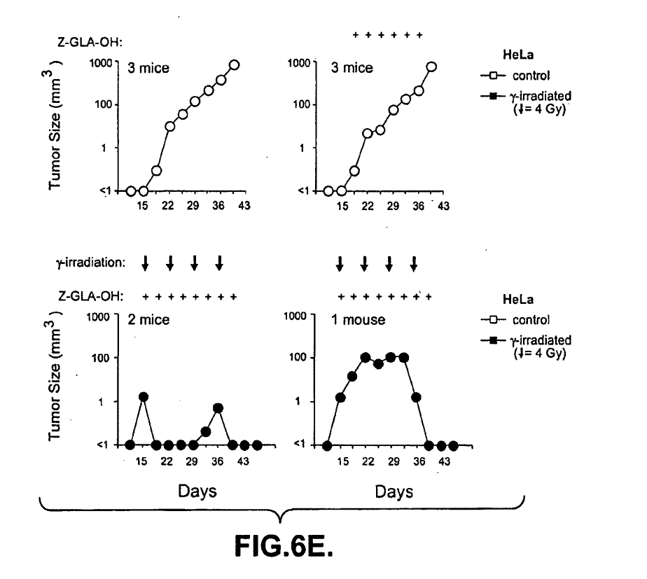

FIG. 6. The Subtilisin inhibitor Z-Gly-Leu-Ala-OH inhibits TPPII and allows efficient radio-sensitization of tumors in vivo. (A) Cleavage of AAF-AMC by partially purified TPPII enzyme, as measured by fluorimetry, in the presence of Z-GLA-OH or butabindide. (B) Tumor growth of 10⁶ EL-4 lymphoma cells in syngeneic C57Bl/6 mice, left untreated (8 mice, empty circles), treated with gamma-irradiation (7 mice, closed circles, 4 Gy doses indicated by arrow) or treated with Z-GLA-OH injection (13.8 mg/kg, indicated with +) as well as gamma-irradiation (8 mice, crossed circles). The data represent the mean tumor size. (C) Tumor growth of 10⁶ EL-4 lymphoma cells in syngeneic C57Bl/6 mice, treated with gamma-irradiation doses of 3 Gy, 2 Gy or 1 Gy in combination with Z-GLA-OH injection (left panel); versus gamma-irradiation doses of 4 Gy or Z-GLA-OH alone and untreated (middle panel). Tumor growth of 10⁶ EL-4 lymphoma cells inoculated into C57Bl/6 mice, treated with gamma-irradiation doses of 3 Gy and Z-GLA-OH at the indicated doses (right panel). In C linear scale was used, to better visualize differences at larger tumor sizes. (D) Tumor growth of 10⁶ Lewis Lung Carcinoma (LLC) cells in syngeneic C57Bl/6 mice, left untreated (open squares) or treated with gamma-irradiation (4 Gy doses indicated by arrow), in the presence (bottom) or absence (top) of Z-GLA-OH. All data points in C and D represent data from at least 4 mice. (F) Tumor growth of 5×10⁶ HeLa cells (human cervical carcinoma) in CB.17 SCID mice, left untreated (open circles), injected with Z-GLA-OH (open squares) or injected with Z-GLA-OH and treated with gamma-irradiation (closed circles, 1.5 Gy per dose, indicated with arrows, closed circles).

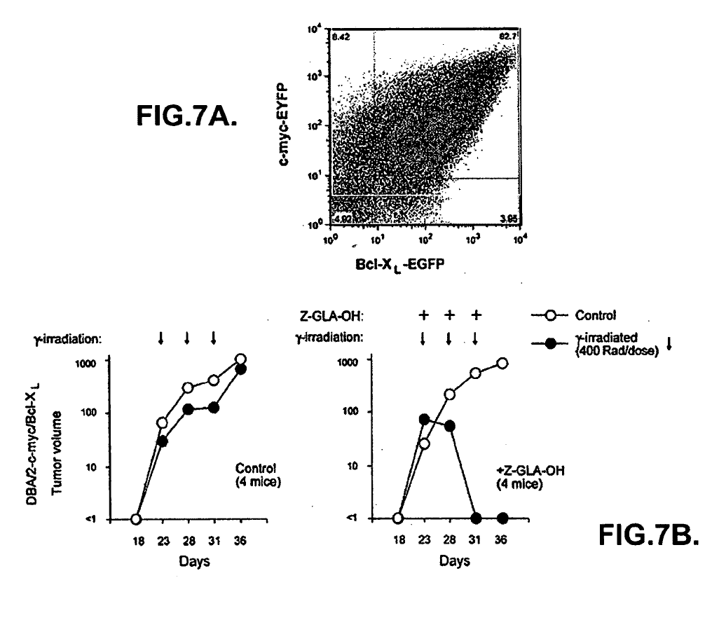

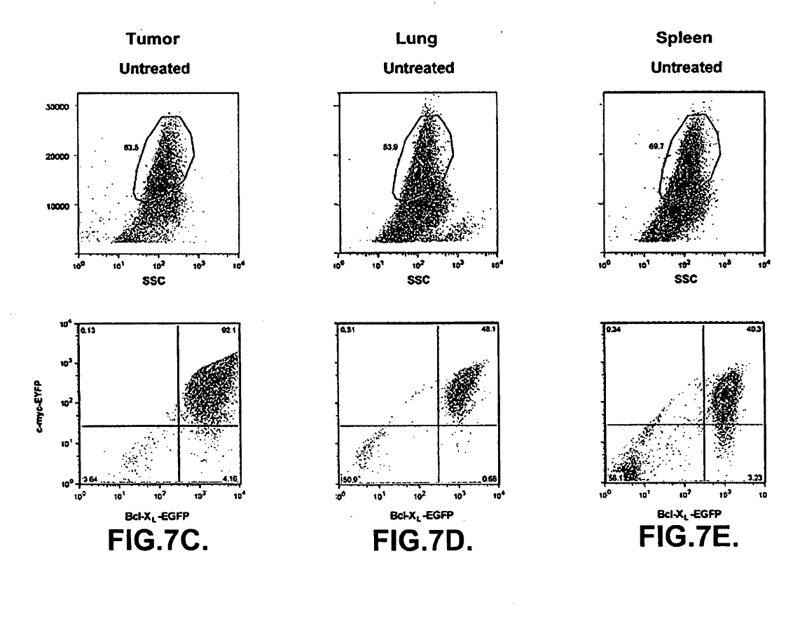

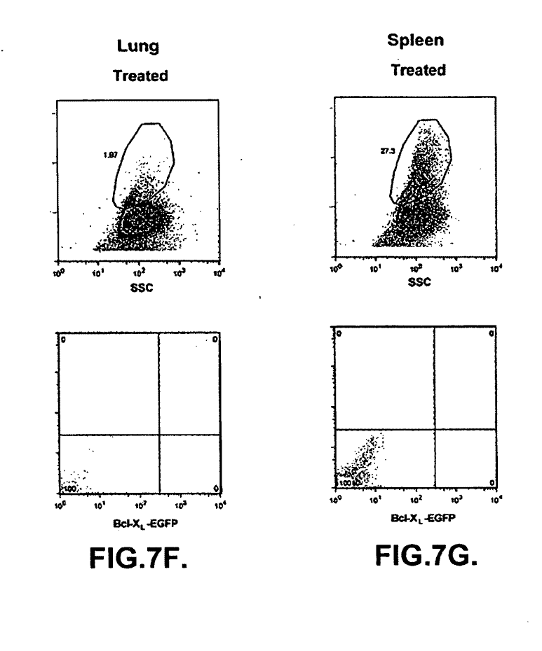

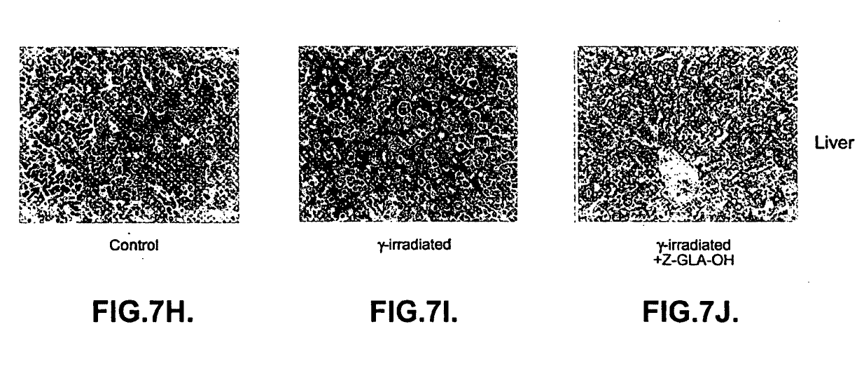

FIG. 7. Radio-sensitization of freshly transformed leukemic cells in vivo.

(A) Flow cytometric analysis of DBA/2 spleen cells 13 days post-transplantation of stem cells transduced with pMSCV-BcI-XL-IRES-E-GFP and pMSCV-c-Myc-IRES-E-YFP. (B) In vivo tumor growth of DBA/2-c-myc/BcI-xL cells in the presence or absence of gamma-irradiation treatment and Z-GLA-OH. (C-G) Flow cytometric detection of vector encoded YFP (c-Myc+) and GFP (BcI-xL+) from DBA-c-Myc/BcI-xL cells in tissues derived from tumour-carrying mice from untreated (C-E) versus treated (F, G) mice (gamma-irradiation and Z-GLA-OH), tissues used were from subcutaneous tumor (C), lung (D, F), and spleen (E, G). Gates indicated in top panels correspond to cells analyzed for GFP/YFP-fluorescence in bottom panels. (H-J) Histological sections of livers from mice inoculated with DBA/2-c-Myc/BcI-xL cells, receiving no treatment (H), gamma-irradiation (I) or both gamma-irradiation and Z-GLA-OH (J). Arrows indicate sinusoids filled with tumor cells.

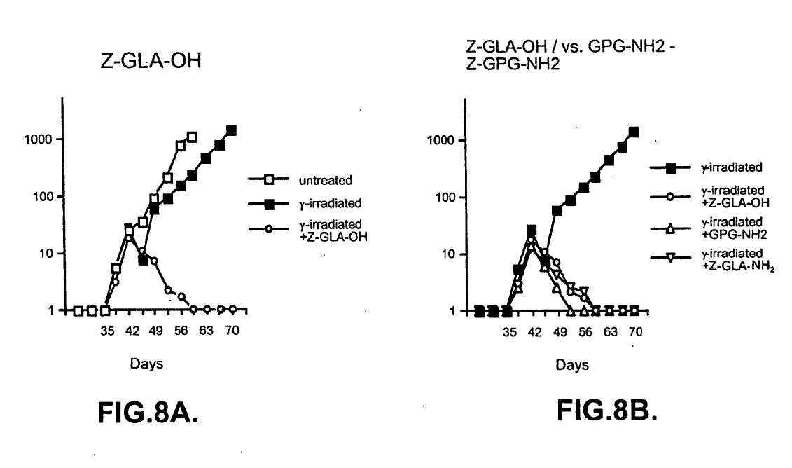

FIG. 8. Strong response to in vivo treatment with GPG-NH₂ or Z-GPG-NH₂ in combination with gamma-irradiation.

Tumour size (vertical axis, mm³) against time (horizontal axis, days) in mice carrying EL-4 tumours treated with gamma-irradiation alone, and treated with gamma-irradiation in combination with each of Z-GLA-OH, GPG-NH₂ and Z-GPG-NH₂.

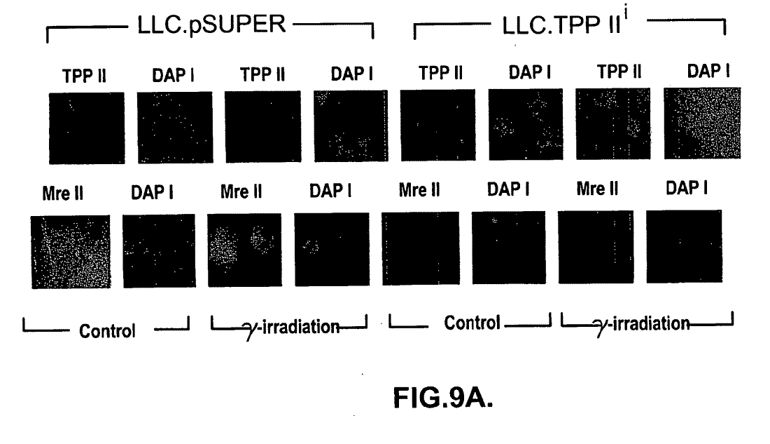

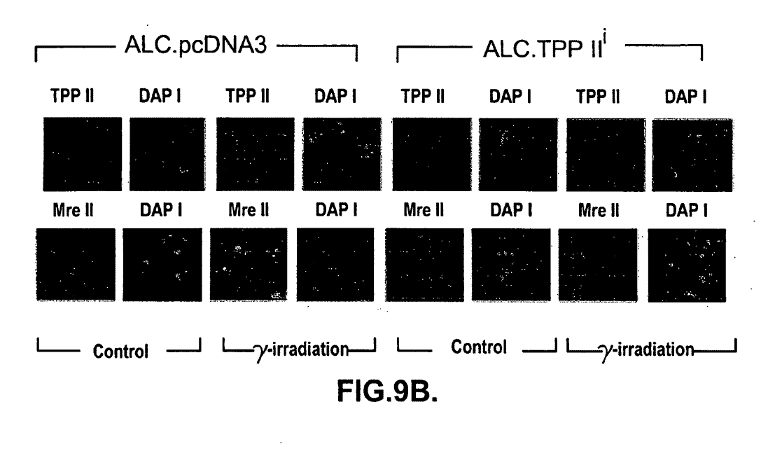

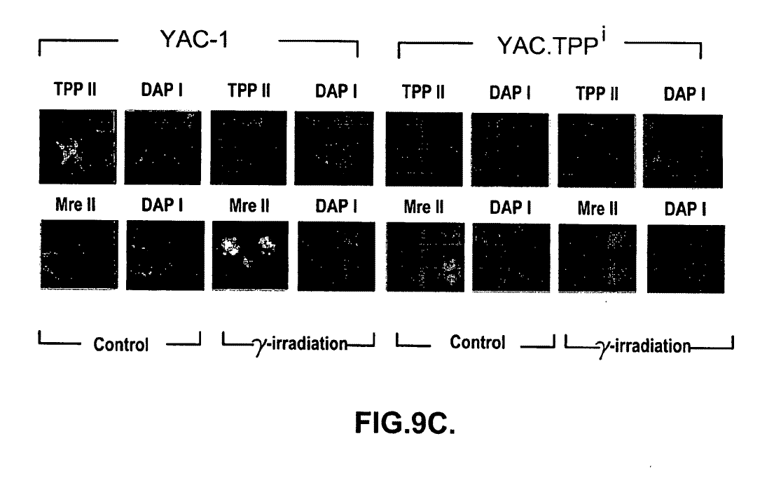

FIG. 9. Inhibition of TPP II affects Mre11 foci formation

The results of further immunocytochemical experiments are shown. Lewis Lung Carcinoma (LLC, A), ALC (B) and YAC-1 (C) cells were stably transfected with pSUPER-TPPIIi, or with empty pSUPER vector, and were exposed to 5 Gy of gamma-irradiation. Immunocytochemical expression of TPPII and Mre11 was measured, as indicated in figure, and DAPI was used for nuclear control staining.

### EXAMPLES

The materials and methods used were as follows.

Cells and Culture Conditions. EL-4 is a Benzpyrene-induced lymphoma cell line derived from the C57BI/6 mouse strain. EL-4.wt and EL-4.TPPIIⁱ are EL-4 cells transfected with the pSUPER vector (Brummelkamp, T R, Bernards, R, Agami, R. A system for stable expression of short interfering RNAs in mammalian cells. Science 2002; 296:550-3), empty versus containing the siRNA directed against TPPII. HeLa cells are human cervical carcinoma cells. YAC-1 is a Moloney Leukemia Virus-induced lymphoma cell line derived from the A/Sn mouse strain. ALC is a T cell lymphoma induced by radiation leukemia virus D-RadLV, derived from the C57Bl/6 mouse strain. For measurement of DNA synthesis cells were seeded into 96-well plates and ³H-Thymidin was added after 16 or 36 hours and incubated for 6 hours before wash. For induction of stress, cells were gamma-irradiated 500-1000 Rad's, or starved by growth in 50%-75% Phosphate Buffered Saline (PBS); and incubated at 37° C. and 5.3% CO₂.

Enzyme Inhibitors. NLVS is an inhibitor of the proteasome that preferentially targets the chymotryptic peptidase activity, and efficiently inhibits proteasomal degradation in live cells. Butabindide is described in the literature (Rose, C, Vargas, F, Facchinetti, P, Bourgeat, P, Bambal, R B, Bishop, P B, et. al. Characterization and inhibition of a cholecystokinin-inactivating serine peptidase. Nature 1996; 380:403-9). Z-Gly-Leu-Ala-OH (Z-GLA-OH) is an inhibitor of Subtilisin (Bachem, Weil am Rhein, Germany), a bacterial enzyme with an active site that is homologous to that of TPPII. Wortmannin is an inhibitor of PIKK (PI3-kinase-related)-family kinases (Sigma, St. Louis, Mo.). All inhibitors were dissolved in DMSO and stored at −20° C. until use.

Protein Purification, Peptidase Assays and Analysis of DNA Fragmentation. 100×10⁶ cells were sedimented and lysed by vortexing in glass beads and homogenisation buffer (50 mM Tris Base pH 7.5, 250 mM Sucrose, 5 mM MgCl2, 1 mM DTT). Cellular lysates were submitted to differential centrifugation where a supernatant from a 1 hour centrifugation at 100,000×g (cytosol) was submitted to 100,000×g centrifugation for 3-5 hours, which sedimented high molecular weight cytosolic proteins/protein complexes. The resulting pellet dissolved in 50 mM Tris Base pH 7.5, 30% Glycerol, 5 mM MgCl2, and 1 mM DTT, and 1 micro-g of high molecular weight protein was used as enzyme in peptidase assays or in Western blotting for TPP II expression. To test the activity of TPP II we used the substrate AAF-AMC (Sigma, St. Louis, Mo.), at 100 micro-M concentration in 100 micro-l of test buffer composed of 50 mM Tri Base pH 7.5, 5 mM MgCl2 and 1 mM DTT. Cleavage activity was measured by emission at 460 nm in a LS50B Luminescence Spectrometer (Perkin Elmer, Boston, Mass.). For analysis of DNA fragmentation cells were seeded in 12-well plates at 10⁶ cells/ml and exposed to 25 micro-M etoposide, a DNA topoisomerase II inhibitor commonly used as an apoptosis-inducing agent, to starvation (50% PBS). Cells were seeded at 10⁶ cells/ml in 12-well plates and incubated for the indicated times, usually 18-24 hours. DNA from EL-4 control and adapted cells was purified by standard chloroform extraction, and 2.5 micro-g of DNA was loaded on 1.8% agarose gel for detection of DNA from apoptotic cells.

Plasmids and Gene Transfection. TPPII siRNA-expressing pSUPER (Brummelkamp, TR, Bernards, R, Agami, R. A system for stable expression of short interfering RNAs in mammalian cells. Science 2002; 296:550-3.) plasmids were constructed as follows. Non-phosphorylated DNA oligomers (Thermo Hybaid, Ulm, Germany) were resuspended to a concentration of 3 micro-g/micro-l. 1 micro-1 of each oligo pair was mixed with 48 micro-l of annealing buffer (100 mM KAc; 30 mM HEPES-KOH pH 7.4; 2 mM MgAc) and heated to 95° C. for 4 min, 70° C. for 10 min, then slowly cooled to room temperature. 2 micro-l of annealed oligomers were mixed with 100 ng of pSUPER plasmid (digested with BglII and HindIII), ligated, transformed, and plated on Amp/LP plates, as previously described (Brummelkamp, T R, Bernards, R, Agami, R. A system for stable expression of short interfering RNAs in mammalian cells. Science 2002; 296:550-3.). Colonies were screened for the presence of inserts by EcoRI-HindIII digestion and DNA sequencing. Annealed oligomer pairs were as follows, for pSUPER-TPPIIⁱ,

forward primer:
5′GATCCCCGATGTATGGGAGAGGCCTTTCAAGAGAAGGCCTCTCCCATA
CATCTTTTTGGAAA-3′;
reverse primer:
5′AGCTTTTCCAAAAAGATGTATGGGAGAGGCCTTCTCTTGAAAGGCCTC
TCCCATACATCGGG-3′.

For generation of stable transfectants, 5×10⁶ cells were washed in PBS, then resuspended into 500 micro-l of PBS in a Bio-Rad gene-pulser and pulsed with 10 micro-g DNA and 250 V at 960 micro-F; and selected by resistance to G418.

Antibodies and Antisera. The following molecules were detected by the antibodies specified: Akt by rabbit anti-Akt serum (Cell Signaling Technology, Beverly, Mass.); Phospho-Akt (Ser 473) by 193H2 rabbit anti-phospho-Akt serum (Cell Signaling Technology, Beverly, Mass.); gamma-H2AX by rabbit anti-gamma-H2AX (Cell Signalling Technology, Beverly, Mass.); Mre11 by polyclonal rabbit anti-human Mre11 (Cell Signalling Technology, Beverly, Mass.); p21 by SX118 (R & D Systems, Minneapolis, Minn.); p53 (R & D Systems, Minneapolis, Minn.); Rae-1 by monoclonal Rat anti-mouse Rae-1, 199215 (R &D Systems, Minneapolis, Minn.); XIAP by monoclonal mouse anti-human XIAP, 117320 (R&D Systems, Minneapolis, Minn.). For detection of TPPII we used chicken anti-TPPII serum (Immunsystem, Uppsala, Sweden). Western blotting was performed by standard techniques. Protein concentration was measured by BCA Protein Assay Reagent (Pierce Chemical Co.). 5 micro-g of protein was loaded per lane for separation by SDS/PAGE unless stated otherwise.

Immunohistochemistry. Cells were attached to glass cover slips through cytospin and fixed in acetone:methanol (1:1) for 1 hour; then the slides were rehydrated in BSS buffer for 1 hour. The first antibody was added and remained for 1 hour until a brief wash in BSS, after which a secondary conjugate (anti-rabbit-FITC) was added and incubated for 1 hour. Then the slides were washed and stained with Hoescht 333258 for 30 min. Finally, the slides were mounted with DABCO mounting buffer and kept at 4° C. until analysis.

Flow Cytometry. For staining of cell surface Rae-1 antigens we incubated 0.5−1.0×10⁶ cells with 50 micro-l of Rae-1 monoclonal antibody 199215 (R &D Systems, Minneapolis, Minn.) at 20 micro-g/ml, and incubated on ice for 30 min. After washing in PBS, we sequentially incubated with Biotinylated Polyclonal Rabbit anti-Rat Ig (Dako Cytomation, Glostrup, Denmark) and Streptavidin-FITC (Pharmingen, San Diego, Calif.), with washing in PBS after each step. Fluorescence was quantified by a FACScalibur. Flow cytometric cell sorting of live cells was performed by incubation of cells for 5 minutes with 2 micro-g/ml of Propidium Iodide (PI) and subsequent sorting into PI⁺ and PI⁻ populations with a FACSvantage.

Tumor Growth Experiments. Tumor cells were washed in PBS and resuspended in a volume of 200 micro-l per inoculate. The cells were then inoculated into the right flank at 10⁶ per mouse and growth of the tumor was monitored by measurement two times per week. The initiation of anti-tumor treatment of the mice was to some extent individualized according to when tumor growth started in each mouse. The mice were irradiated with 4 Gy prior to tumor inoculation in order to inhibit anti-tumor immune responses. The tumor volume was calculated as the mean volume in mice with tumors growth, according to (a₁×a₂×a₃)/2 (the numbers aᵢ denote tumor diameter, width and depth). The time of first palpation varied between different mice, although the general pattern of growth was similar in virtually all of the mice. In most diagrams a log-scale is used to better visualize the therapeutic effects against small tumors, i.e. the presence of complete rejections. For inhibition of TPPII in vivo we made intraperitoneal injections with 13.8 mg per kg of body weight (14 micro-l of a 50 mM solution/mouse) of the Subtilisin inhibitor Z-Gly-Leu-Ala-OH (Z-GLA-OH, Bachem, Weil am Rhein, Germany) twice per week, diluted into 200 micro-l PBS. All gamma-irradiations were full body exposures.

Retroviral transduction and transplantation. With reference to Example 7, the sequence for c-Myc was amplified from human cDNA (brain) by PCR using the following primers: 5′ACGTGAATTCCACCATGCCCCTCAACGTTAGCTTC and 3′ACGTCTCGAGCTTACGCACAAGAGTTCCGTAG and inserted in the EcoRI site of the retroviral expression vector pMSCV-IRES-EYFP. hBcl-x_{L} was excised from the pLXIN-hBcl -x_{L} (Djerbi, M., Darreh-Shori, T., Zhivotovsky, B. & Grandien, A. Characterization of the human FLICE-inhibitory protein locus and comparison of the anti-apoptotic activity of four different flip isoforms. Scand J. Immunol. 54, 180-9, 2001) and inserted into the EcoRI site of the pMSCV-IRES-EGFP. Production of retroviral particles, enrichment and transduction of hematopoietic stem cells and transplantation was performed as described previously (Nyakeriga, A.M., Djerbi, M., Malinowski, M.M. & Grandien, A. Simultaneous expression and detection of multiple retroviral constructs in haematopoietic cells after bone marrow transplantation. Scand J Immunol. 61, 545-50, 2005). Briefly, retroviral vectors were transiently transfected into Phoenix-Eco packaging cells using the LipofectAMINE 2000 Reagent (Invitrogen, Life Technologies Inc., Paisley, UK) and viral supernatants containing viral particles were harvested and used to transduce lineage negative cells obtained from bone marrow of 5-fluorouracil treated mice. These cells were thereafter injected into lethally irradiated recipient mice. Between 7 and 14 days after transplantation, the mice developed an acute myeloid leukaemia-like disease. Cells from spleen of such mice could be grown in vitro in regular RPMI medium supplemented with, glutamin and fetal calf serum.

Detection of GFP and YFP expression was performed using a Cyan™ ADP cytometer (Dako, Glostrup, Denmark) where after excitation at 488 nm, a 525-nm long-pass dichroic mirror was used to initially separate the signals followed by a 510/21-nm bandpass filter for detection of EGFP and a 550/30-nm band pass filter for EYFP. Data were analyzed using FlowJo software (Tree Star, Inc., San Carlos, Calif.).

Abbreviations list: ATM, Ataxia Telangiectasia Mutated; BRCT, BRCA C-terminal repeat; NLVS, 4-hydroxy-5-iodo-3-nitrophenylacetyl-Leu-Leu-Leu-vinyl sulphone; PI, Propidium Iodide; PIKKs, Phosphoinositide-3-OH-kinase-related kinases; TPPII, Tripeptidyl-peptidase II; FA, 3-(2-furyl)acryloyl; YFP, Yellow Fluorescent Protein, GFP, Green Fluorescent Protein;

Standard abbreviations are used for chemicals and amino acids herein.

| Abbreviation | | Alternative abbreviation |
|---|---|---|
| A | Alanine | Ala |
| R | Arginine | Arg |
| N | Asparagine | Asn |
| D | Aspartic acid | Asp |
| C | Cysteine | Cys |
| E | Glutamic Acid | Glu |
| Q | Glutamine | Gln |
| G | Glycine | Gly |
| H | Histidine | His |
| I | Isoleucine | Ile |
| L | Leucine | Leu |
| K | Lysine | Lys |
| M | Methionine | Met |
| F | Phenylalanine | Phe |
| P | Proline | Pro |
| S | Serine | Ser |
| T | Threonine | Thr |
| W | Tryptophan | Trp |
| Y | Tyrosine | Tyr |
| V | Valine | Val |

The invention also makes use of several unnatural alpha-amino acids.

| Abbreviation | | SIDE CHAIN |
|---|---|---|
| Abu | 2-aminobutyric acid | CH₂CH₃ |
| Nva | norvaline | CH₂CH₂CH₃ |
| Nle | norleucine | CH₂CH₂CH₂CH₃ |
| | tert-butyl alanine | CH₂C(CH₃)₃ |
| | alpha-methyl leucine | (CH₃)(CH₂C(CH₃)CH₃) |
| | 4,5-dehydro-leucine | CH₂C(═CH₂)CH₃ |
| | allo-isoleucine | CH(CH₃)CH₂CH₃ |
| | alpha-methyl valine | (CH₃)CH(CH₃)(CH₃) |
| | tert-butyl glycine | C(CH₃)₃ |
| | 2-allylglycine | CH₂CH═CH₂ |
| Orn | Ornithine | CH₂CH₂CH₂NH₂ |
| Dab | alpha,gamma-diaminobutyric | CH₂CH₂NH₂ |
| | acid | |
| | 4,5-dehydro-lysine | CH₂CH═CHCH₂NH₂ |

### Example 1 and FIG. 1

### Gamma-Irradiation-Induced Cell Cycle Arrest Depends on TPPII Expression

Since TPPII expression is increased by several types of stress we tested whether this was controlled by PIKKs. By Western blotting analysis of the T cell lymphoma line EL-4 with TPPII anti-serum we found that TPPII expression was increased by gamma-irradiation. Further, this increase was not present in gamma-irradiated EL-4 cells treated with 1 micro-M wortmannin, a PIKK inhibitor, which instead reduced TPPII expression (FIG. 1A). Treatment with NLVS, a proteasomal inhibitor, inhibited down regulation of TPPII in wortmannin treated gamma-irradiated EL-4 cells, suggesting that TPPII is degraded by the proteasome in the absence of PIKK signaling (FIG. 1A). To further study whether TPPII had any role in cellular responses mediated by PIKKs, we generated stable EL-4 transfectants expressing siRNA against TPPII, encoded by the pSUPER vector (denoted EL-4.TPPIIⁱ, [Brummelkamp, T R, Bernards, R, Agami, R. A system for stable expression of short interfering RNAs in mammalian cells. Science 2002; 296:550-3.]). EL-4.TPPIIⁱ cells had both inhibited expression and activity of TPPII, in comparison to EL-4.wt cells (transfected with empty pSUPER vector, FIG. 1B). To trigger a cellular stress response where members of the PIKK family members control signal transduction, we used gamma-irradiation (5 Gy). TPPII was previously reported as a soluble cytosolic peptidase (Reits, E, Neijssen, J, Herberts, C, Benckhuijsen, W, Janssen, L, Drijfhout, J W, et. al. A major role for TPPII in trimming proteasomal degradation products for MHC class I antigen presentation. Immunity 2004; 20:495-506), but we here found rapid translocation of TPPII into the nucleus of gamma-irradiated EL-4 cells (FIG. 1C). This was evident already 1 hour following gamma-irradiation exposure of EL-4 cells, as detected by immunohistochemical analysis of TPPII. A similar response was observed in ALC and YAC-1 lymphoma as well as Lewis Lung Carcinoma (LLC) cells.

Activation of PIKKs is required to halt DNA synthesis in response to DNA damage (Bakkenist, C J, Kastan M B. Initiating cellular stress responses. Cell 2004; 118:9-17) (McKinnon, P J. ATM and ataxia telangiectasia. EMBO Rep. 2004; 5:772-6). We observed that DNA synthesis was inhibited in gamma-irradiated EL-4.wt control, but we found high levels of gamma-irradiation-resistant DNA synthesis in EL-4.TPPIIⁱ cells up to 36 hours after exposure (as measured by ³H-Thymidin incorporation, FIG. 1D). These data suggested that TPPII was important to halt DNA synthesis of EL-4 cells in response to gamma-irradiation. EL-4.TPPIIⁱ cells arrested almost uniformly in G2/M after exposure to gamma-irradiation, whereas EL-4.wt control cells showed both G1 and G2/M arrest, suggesting an absence of a G1/S checkpoint in EL-4.TPPIIⁱ cells (FIG. 1E). However, initial detection of DNA damage was still present in gamma-irradiated EL-4.TPPIIⁱ cells, as measured by western blotting of gamma-H2AX (Ser139-phosphorylated H2AX, FIG. 1F). H2AX is phosphorylated in response to ATM activation, which triggers the formation of DNA repair foci (Bakkenist, C J, Kastan M B. Initiating cellular stress responses. Cell 2004; 118:9-17). Thus, TPPII is rapidly translocated into the nucleus following gamma-irradiation-exposure, and required to efficiently halt DNA synthesis in EL-4 cells, but not for phosphorylation of H2AX.

### Example 2 and FIG. 2

### Failure to Stabilize p53 in Cells with Inhibited TPPII Expression

The transcription factor p53 initiates cell cycle arrest in response to many types of stress, and its expression is controlled by direct phosphorylation by PIKKs. By Western blotting analysis in cellular lysates of gamma-irradiated EL-4.wt cells, we found increased levels of p53, whereas those of EL-4.TPPIIⁱ cells showed low levels (FIG. 2A). However, treatment with NLVS increased p53 expression of gamma-irradiated EL-4.TPPIIⁱ cells, suggesting that p53 was still synthesized but degraded by the proteasome in EL-4.TPPIIⁱ cells. p21, a transcriptional target of p53, was weakly expressed in EL-4.TPPIIⁱ cells following exposure to gamma-irradiation, compared to EL-4.wt control cells (FIG. 2B). Further, EL-4.pcDNA-TPPII cells that stably over-express TPPII, showed increased levels of p53 following exposure to gamma-irradiation in comparison to EL-4.pcDNA3 cells (Wang, E W, Kessler, B M, Borodovsky, A, Cravatt, B F, Bogyo, M, Ploegh, H L, et. al. Integration of the ubiquitin-proteasome pathway with a cytosolic oligopeptidase activity. Proc Natl Acad Sci USA. 2000; 97:9990-5.) (FIG. 2C). To test if p53 and TPPII were physically linked we next performed co-immuno-precipitation experiments using an anti-serum directed against the N-terminus of p53, followed by western blot analysis for TPPII. In p53 immuno-precipitates from lysates of EL-4-pSUPER cells we detected TPPII; levels that were increased by gamma-irradiation (FIG. 2D, top). This was not observed in lysates from EL-4.TPPIIⁱ cells or from lysates of EL-4.wt cells treated with 1 micro-M wortmannin (FIG. 2D). These data supported a gamma-irradiation-induced physical link between TPPII and p53. We found that p53 expression was also TPPII-dependent in gamma-irradiated YAC-1 and ALC lymphoma cells, where virtually no p53 was detectable following stable expression of pSUPER-TPPIIⁱ (FIG. 2E). We failed to find expression of p53 in Lewis Lung Carcinoma (LLC) cells (FIG. 2E). We noted substantial levels of p53 in some of our control tumor cell lines also prior to exposure to gamma-irradiation, a phenomenon in line with the frequently up-regulated DNA damage response in transformed cells (Bartkova, J, Horejsi, Z, Koed, K, Kramer, A, Tort, F, Zieger, K, et. al. Activation of the DNA damage checkpoint and genomic instability in human precancerous lesions. Nature 2005; 434:907-13) (Bartkova, J, Horejsi, Z, Koed, K, Kramer, A, Tort, F, Zieger, K, et. al. DNA damage response as a candidate anti-cancer barrier in early human tumorigenesis. Nature 2005; 434:864-70). We concluded that TPPII expression was required for efficient stabilization of p53.

### Example 3 and FIG. 3

### TPPII Controls Activation of Several Pathways that Depend on PIKK Signaling

Since TPPII expression was a requirement for stabilization of p53 we tested also other stress-induced pathways that depend on PIKK signaling (Gasser, S, Orsulic, S, Brown, E J, Raulet, D H. The DNA damage pathway regulates innate immune system ligands of the NKG2D receptor. Nature 2005; 436:1186-90) (Viniegra, J G, Martinez, N, Modirassari, P, Losa, J H, Parada Cobo, C, Lobo, V J, et. al. Full activation of PKB/Akt in response to insulin or ionizing radiation is mediated through ATM. J Biol. Chem. 2005; 280:4029-36) (Feng, J, Park, J, Cron, P, Hess, D, Hemmings, B A. Identification of a PKB/Akt hydrophobic motif Ser-473 kinase as DNA-dependent protein kinase. J Biol Chem 2004; 279:41189-96) (Sarbassov, D D, Guertin, D A, Ali, S M, Sabatini, D M. Phosphorylation and regulation of Akt/PKB by the rictor-mTOR complex. Science 2005; 307:1098-101), by comparing their status in EL-4.wt versus EL-4.TPPIIⁱ cells. Ser473 phosphorylation of Akt kinase requires PIKK signaling by ATM, DNA-PK or mTOR, the mechanistic details are debated (Viniegra, J G, Martinez, N, Modirassari, P, Losa, J H, Parada Cobo, C, Lobo, V J, et. al. Full activation of PKB/Akt in response to insulin or ionizing radiation is mediated through ATM. J Biol. Chem. 2005; 280:4029-36) (Feng, J, Park, J, Cron, P, Hess, D, Hemmings, B A. Identification of a PKB/Akt hydrophobic motif Ser-473 kinase as DNA-dependent protein kinase. J Biol Chem 2004; 279:41189-96) (Sarbassov, D D, Guertin, D A, Ali, S M, Sabatini, D M. Phosphorylation and regulation of Akt/PKB by the rictor-mTOR complex. Science 2005; 307:1098-101). We detected substantial levels of phospho-Ser473-Akt in lysates of EL-4.wt cells. However, EL-4.TPPIIⁱ cells displayed very low levels of phospho-Ser473-Akt, whereas total expression of Akt was similar (FIG. 3A). In addition, we find increased Ser473-phosphorylation of Akt in EL-4.pcDNA3-TPPII, in comparison to EL-4.pcDNA3 control cells further supporting that TPPII expression controls Akt-Ser473-phosphorylation (FIG. 3B). Akt kinase is important for transduction of cell survival signals, and is over-activated in many tumors. In normal medium (5% serum) EL-4.TPPIIⁱ cells showed an increased rate of proliferation, compared to EL-4.wt, but also an increased accumulation of dead cells (FIG. 3C). Further, by lowering serum concentrations to 1% this accumulation was accelerated, compared to EL-4.wt cells, suggesting that cell survival mechanisms were impaired in the absence of TPPII (FIG. 3C). In addition, EL-4.pcDNA3-TPPII cells were able perform limited growth in 0.5% serum, which EL-4.pcDNA3 cells did not (FIG. 3D). These phenotypes indicate that TPPII expression is important for Akt Ser473 phosphorylation and cell survival during in vitro culture. XIAP, a direct substrate of Akt kinase (Dan, H C, Sun, M, Kaneko, S, Feldman, R I, Nicosia, S V, Wang, H G, et. al. Akt phosphorylation and stabilization of X-linked inhibitor of apoptosis protein (XIAP). J Biol Chem. 2004; 279:5405-12), is a member of the IAP family of molecules; endogenous caspase inhibitors commonly over-expressed in tumor cells. Up-regulation of TPPII causes increased expression of c-IAP-1 and XIAP molecules in EL-4.pcDNA3-TPPII cells. By treatment with etoposide we found that expression of XIAP was substantially higher in EL-4.wt cells, compared to EL-4.TPPIIⁱ cells, with a slower rate of degradation (FIG. 3E). Further, activation of ATM and ATR kinases mediate expression of NKG2D ligands, thereby allowing the immune system to detect cells with an ongoing DNA damage response (Gasser, S, Orsulic, S, Brown, E J, Raulet, D H. The DNA damage pathway regulates innate immune system ligands of the NKG2D receptor. Nature 2005; 436:1186-90). By flow cytometric measurements we detected expression of Rae-1 on EL-4.wt cells, whereas minor amounts of Rae-1 expression were detected on EL-4.TPPIIⁱ cells (FIG. 3F). We failed to detect expression of Rae-1 ligands on ALC lymphoma cells, but analysis of mouse YAC-1 lymphomas also showed that Rae-1 expression was dependent on TPPII expression, since stable pSUPER-TPPIIⁱ transfectants (YAC-1.TPPIIⁱ) express minor levels of Rae-1 ligands at the cell surface (FIG. 3G). These data show that that several stress-induced pathways activated by PIKKs require TPPII expression.

### Example 4 and FIG. 4

### A BRCT-Like Motif of TPPII Required for p53 Stabilization in Response to Gamma-Irradiation

BRCA C-terminal repeat (BRCT)-domains are often contained within proteins controlling DNA damage signaling pathway, where they control interactions with ATM substrates (Bork, P, Hofmann, K, Bucher, P, Neuwald, A F, Altschul, S F, Koonin, E V. A superfamily of conserved domains in DNA damage-responsive cell cycle checkpoint proteins. FASEB J. 1997; 11:68-76) (Manke, I A, Lowery, D M, Nguyen, A, Yaffe, M B. BRCT repeats as phosphopeptide-binding modules involved in protein targeting. Science 2003; 302:636-9) (Yu, X, Chini, C C, He, M, Mer, G, Chen, J. The BRCT domain is a phospho-protein binding domain. Science 2003; 302:639-42). We found one region of TPPII centered around the GG-doublet at position 725 which matched most, but not all, requirements of a BRCT motif (FIG. 4A). We performed site-directed mutagenesis of the characteristic Gly-Gly-doublet present in many BRCT sequences (labeled *, FIG. 4A), mutating it into Gly-Glu in our pcDNA3-TPPII vector. To allow expression of this plasmid in EL-4.TPPIIⁱ cells, we inserted 3 silent mutations in the 3′ region of TPPII among the nucleotides that interact with the pSUPER-TPPIIⁱ-encoded siRNA (this plasmid was denoted TPPII^{wt}), in addition to the mutation in position 725 (denoted TPPII^{wt}/G725E). We found that both TPPII^{wt} as well as TPPII^{wt}/G725E mutant molecules were stably expressed in EL-4 cells co-transfected with pSUPER-TPPIIⁱ (FIG. 4B). Further, the expression of p53 was analyzed in EL-4.TPPII^{wt} and EL-4.TPPII^{wt}/G725E transfectant cells exposed to gamma-irradiation. We found that EL-4.TPPII^{wt}/G725E cells showed much reduced expression of p53, compared to EL-4.TPPII^{wt} control cells (FIG. 4C). In addition, we failed to detect TPPII in p53-immunoprecipitates from lysates of EL-4.TPPII^{wt}/G725E cells, both in the presence and absence of gamma-irradiation, whereas TPPII was detected using EL-4.TPPII^{wt} control cells (FIG. 4D). We concluded that TPPII possesses a BRCT-like domain important for DNA damage signaling.

Regulatory factors are co-localized at sites of DNA damage, to allow the activation of downstream responses (Al Rashid, S T, Dellaire, G, Cuddihy, A, Jalali, F, Vaid, M, Coackley, C, et. al. Evidence for the direct binding of phosphorylated p53 to sites of DNA breaks in vivo. Cancer Res. 2005; 65:10810-21) (Lisby, M, Barlow, J H, Burgess, R C, Rothstein, R. Choreography of the DNA damage response: spatiotemporal relationships among checkpoint and repair proteins. Cell. 2004; 118:699-713). A possible reason behind the failure of p53 stabilization in cells with inhibited TPPII expression is that p53 fails to be recruited to such sites. We examined the presence of DNA repair foci components in p53 immuno-precipitates from EL-4.wt versus EL-4.TPPIIⁱ cells. We detected ATM in p53-immuno-precipitates from EL-4.wt, but not from EL-4.TPPIIⁱ cells, as measured by Western blotting (FIG. 4 E). We also detected DNA repair foci proteins 53BP1 and Mre11 among p53-linked proteins upon gamma-irradiation, in EL-4.wt, but not in EL-4.TPPIIⁱ cells (FIG. 4F, G). Further, NLVS-treated EL-4.TPPIIⁱ cells also failed to show ATM, 53BP1 and Mre11 in p53-immunoprecipitates (FIG. 4E-G). The fact that p53 and ATM are found in proximity to DNA repair foci components is in line with that certain p53 isoforms accumulate at these foci, where they may interact with ATM kinase (Al Rashid, S T, Dellaire, G, Cuddihy, A, Jalali, F, Vaid, M, Coackley, C, et. al. Evidence for the direct binding of phosphorylated p53 to sites of DNA breaks in vivo. Cancer Res. 2005; 65:10810-21). Our data support that a physical link between p53 and ATM, as well as DNA repair foci components 53BP1 and Mre11, requires TPPII.

### Example 5 and FIG. 5

### TPPII Expression Controls Gamma-Irradiation Resistance of EL-4 Tumors In Vivo

PIKKs are possible target molecules for the development of novel cancer therapies (Choudhury, A, Cuddihy, A, Bristow, R G. Radiation and new molecular agents part I: targeting ATM-ATR checkpoints, DNA repair, and the proteasome. Semin Radiat Oncol 2006; 16:51-8). To address whether TPPII-mediated growth regulation was important for in vivo tumor growth we inoculated 10⁶ EL-4.wt control or EL-4.TPPIIⁱ cells into syngeneic C57Bl/6 mice. We found that both EL-4.wt and EL-4.TPPIIⁱ cells established tumors and grew at an approximately equal rate, suggesting when considered in isolation that TPPII was not important for growth of EL-4 tumors in vivo (FIG. 5A, B, panels labeled control). However, we also treated mice carrying either tumors of EL-4.wt or EL-4.TPPIIⁱ cells with 2-4 doses of 4 Gy (400 Rad's) gamma-irradiation. We found that this had minor effects on tumor size after inoculation with 10⁶ EL-4.wt cells that continued to grow despite gamma-irradiation (FIG. 5 A, gamma-irradiation indicated with arrow). In contrast, mice carrying tumors of EL-4.TPPIIⁱ cells responded to gamma-irradiation treatment with complete regression of established tumors (FIG. 5B). These data resembled those obtained with tumors of EL-4.ATMⁱ or EL-4.TPPII^{wt}/G725E cells, since these also failed to resist gamma-irradiation in vivo (FIG. 5C, D). The data support TPPII as a target to increase in vivo gamma-irradiation susceptibility of tumor cells.

### Example 6 and FIG. 6

### Tri-Peptide-Based TPPII Inhibitors Radio-Sensitize Tumors In Vivo

TPPII is a Subtilisin-type Serine peptidase, with a catalytic domain that is homologous to bacterial Subtilisins (Tomkinson, B, Wernstedt, C, Hellman, U, Zetterqvist, O. Active site of tripeptidyl peptidase II from human erythrocytes is of the subtilisin type. Proc Natl Acad Sci USA. 1987; 84:7508-12). We found that the tri-peptide Subtilisin inhibitor Z-Gly-Leu-Ala-OH (Z-GLA-OH) efficiently inhibited TPPII, with a Kᵢ50 of about 10 nM, slightly less efficient than observed for butabindide (which has a Kᵢ50 of 7 nM), as observed by inhibited TPPII cleavage of the substrate AAF-AMC (FIG. 6A). Moreover, Z-GLA-OH was relatively stable in serum.

To test the effects of catalytic TPPII inhibition during tumor gamma-irradiation in vivo, we exposed C57Bl/6 mice with established EL-4 tumors to gamma-irradiation doses of 4 Gy (one dose/week) and injections with Z-GLA-OH twice weekly (13.8 mg/kg body weight). Weekly gamma-irradiation doses of 4 Gy had minor effects on growth of established EL-4 tumors in C57Bl/6 control mice. In contrast, following injection with Z-GLA-OH we observed complete tumor regression after 3-4 doses of 400 Rad gamma-irradiation in all mice tested (FIG. 6B). When these tumors were no longer palpable the treatment was cancelled, and no re-growth of tumors was observed for the entire period of observation (over 3 months). Titrations of the gamma-irradiation dose, in the presence of Z-GLA-OH injection, also showed complete regression of EL-4 tumors in mice exposed to doses of 3 Gy, whereas lower doses of gamma-irradiation reduced tumor growth also with some complete rejections (2 Gy, 2 out of 5 mice; 1 Gy, 1 out of 4, FIG. 6C). Titrations of the Z-GLA-OH compound showed complete tumor rejection in response to gamma-irradiation in most mice following inoculations with 6.9 mg/kg of Z-GLA-OH (3 out of 4), whereas 3.5 mg/kg and lower doses gave partial effects in terms of tumor regression (2 out of 4; using 3 Gy gamma-irradiation doses; FIG. 6C, right panel).

One common reason behind tumor therapy resistance, including in vivo resistance to gamma-irradiation, is p53 mutations (EI-Deiry, W S. The role of p53 in chemosensitivity and radiosensitivity. Oncogene 2003; 22:7486-95). To test whether also p53-mutated tumors responded to gamma-irradiation in the presence of TPPII inhibitors we similarly inoculated 10⁶ Lewis Lung Carcinoma (LLC) cells in syngeneic C57B1/6 mice. We found that LLC tumors were virtually insensitive to repeated gamma-irradiation doses of 4 Gy, and Z-GLA-OH only (in the absence of gamma-irradiation) gave no effect (FIG. 6D). In contrast, we observed complete regression of established LLC tumors to gamma-irradiation in mice injected with Z-GLA-OH (FIG. 6D). We found that a protected di-peptide Z-GL-OH, was ineffective both in terms of TPPII inhibition and radio-sensitization of LLC tumors, whereas the N-terminal protective Z-group was not strictly required for anti-tumor effects in vivo. TPPII is an evolutionary conserved enzyme with an identity of 96% at the amino acid level between human and mouse, and we observed strong tumor regression also of human HeLa cervical carcinoma cells in Z-GLA-OH-treated SCID mice in response to gamma-irradiation (FIG. 6E). A reduced dose of gamma-irradiation (1.5 Gy/dose) was used, since SCID mice have substantially reduced radio-resistance.

Toxicity studies show that Z-GLA-OH had minor effects in vivo as single agent in doses up to 100 mg/kg, in a preliminary study. Furthermore, our mice survived for an extended period of time after the study. Since the gamma-irradiation protocols used here were exclusively whole body exposures, all tissues where Z-GLA-OH was distributed were exposed to gamma-irradiation and Z-GLA-OH in combination. This suggests manageable toxicity for the combined treatment.

### Example 7 and FIG. 7

### Radio-Sensitization of Freshly Transformed Leukemic Cells In Vivo

To establish tumor cells that more resemble primary tumors we used a retroviral expression system with two separate vectors encoding c-Myc and Bcl-x_{L} (pMSCV-Bcl-x_{L} IRES-EGFP and pMSCV-c-Myc-IRES-EYFP). DBA/2 bone-marrow cells were retrovirally infected with these Bcl-x_{L}- and c-Myc-expressing vectors and transplanted into gamma-irradiated syngeneic mice. Vector-encoded Green Fluorescence Protein (GFP) versus Yellow Fluorescence Protein (YFP) allowed monitoring of retroviral gene expression (Nyakeriga, A.M., Djerbi, M., Malinowski, M.M. & Grandien, A. Simultaneous expression and detection of multiple retroviral constructs in haematopoietic cells after bone marrow transplantation. Scand J. Immunol. 61, 545-50, 2005). 7-14 days post-transplantation we observed a massive accumulation of YFP⁺/GFP⁺ myeloid (CD11b⁺Gr1⁺) blasts in the spleen and bone-marrow (shown for spleen, FIG. 7 A). We inoculated these DBA/2-c-Myc/Bcl-x_{L} cells subcutaneously into syngeneic DBA/2 mice, and we observed palpable tumors after about 3 weeks that grew to sizes exceeding 1000 mm³ within an additional 2-3 weeks (FIG. 7 B). In all mice inoculated with DBA/2-c-Myc/Bcl-x_{L} cells we found tumor dissemination into the liver, as observed by histological analysis of fixed organs (FIG. 7 H). These malignant cells were also detected by flow cytometry showing YFP⁺/GFP⁺ cells in the spleen, lung and liver, using the cells from the primary tumor as control (FIG. 7 C-G). By treatment with gamma-irradiation (4 Gy/dose, 1 dose/week), we observed slightly reduced growth but the DBA/2-c-Myc/Bcl-x_{L} tumors still reached sizes exceeding 1000 mm³ with a delay of less than one week, also with the presence of liver metastasis (FIG. 7 B). In contrast, mice with established DBA/2-c-Myc/Bcl-x_{L} tumors receiving Z-GLA-OH (13.8 mg/kg body weight) had complete tumor regression in response to 4 Gy-doses of gamma-irradiation (FIG. 7 B). Further, we failed to find tumor cells in either lung, spleen or liver in these Z-GLA-OH-treated mice (FIG. 7 F, G, J). Gamma-irradiation was required for this treatment response, since no reduction of tumor size was observed in mice receiving Z-GLA-OH only (FIG. 7 B). These data support that the radio-sensitizing effect observed from Z-GLA-OH is unlikely to depend on specific tumor defects, but can be observed in cells freshly transformed by a simple two-hit strategy, deregulating proliferation and apoptosis.

### Example 8

### In Vitro Testing of Di- and Tri-Peptides and Derivatives

Table 1 contains in vitro data, in fluorometric units which are arbitrary but relative, for the inhibition of cleavage of AAF-AMC (H-Ala-Ala-7-amido-4-methylcoumarin) by compounds at several concentrations. Some beneficial effect is seen for most of the compounds tested.

TPP II protein was enriched, and then a TPP II-preferred fluorogenic substrate AAF-AMC was used. 100×10⁶ cells were sedimented and lysed by vortexing in glass beads and homogenisation buffer (50 mM Tris Base pH 7.5, 250 mM Sucrose, 5 mM MgCl₂, 1 mM DTT). Cellular lysates were subjected to differential centrifugation; first the cellular homogenate was centrifuged at 14,000 rpm for 15 min, and then the supernatant was transferred to ultra-centrifugation tubes. Next the sample was ultra-centrifugated at 100,000×g for 1 hour, and the supernatant (denoted as cytosol in most biochemical literature) was subjected to 100,000×g centrifugation for 5 hours, which sedimented high molecular weight cytosolic proteins/protein complexes. The resulting pellet dissolved in 50 mM Tris Base pH 7.5, 30% Glycerol, 5 mM MgCl₂, and 1 mM DTT, and 1 ug of high molecular weight protein was used as enzyme in peptidase assays.

To test the activity of TPP II we used the substrate and AAF-AMC (Sigma, St. Louis, Mo.), at 100 uM concentration in 100 ul of test buffer composed of 50 mM Tri Base pH 7.5, 5 mM MgCl₂ and 1 mM DTT. To stop reactions we used dilution with 900 ul 1% SDS solution. Cleavage activity was measured by emission at 460 nm in a LS50B Luminescence' Spectrometer (Perkin Elmer, Boston, Mass.).

FA=3-(2-furyl)acryloyl; PBS=phosphate-buffered saline. The text (Z, FA, H, etc.) at the start of each compound name is the substituent at the N-terminus; H indicates that the N-terminus is free NH₂. The text (OH, NBu, etc.) at the end of each compound name is the substituent at the C-terminus; OH indicates that the C-terminus is free CO₂H.

TABLE 1

| | 100 | 10 | 1 | | | | |
|---|---|---|---|---|---|---|---|
| Compound | uM | uM | uM | 100 nM | 10 nM | 1 nM | 0 |
| Z-GL-OH | 23.14 | 23.60 | 24.18 | 34.6 | 34.07 | 44.53 | 49.55 |
| (comparative) | 24.99 | 24.72 | 24.4 | 33.02 | 33.85 | 44.21 | 49.82 |
| | 23.69 | 24.59 | 24.29 | 34.6 | 34.38 | 43.62 | 49.51 |
| mean | 23.94 | 24.30 | 24.29 | 34.07 | 34.1 | 44.12 | 49.63 |
| Z-GLG-OH | 14.44 | 17.49 | 23.79 | 31.49 | 34.4 | 43.42 | 48.58 |
| | 15.02 | 17.58 | 24.85 | 28.64 | 34.16 | 44.02 | 49.03 |
| | 15.8 | 17.44 | 24.63 | 26.13 | 34.27 | 43.73 | 49.2 |
| mean | 15.09 | 17.50 | 24.42 | 28.75 | 34.28 | 43.72 | 48.94 |
| Z-GGA-OH | 15.5 | 16.65 | 21.37 | 24.27 | 36.01 | 43.42 | 51.19 |
| | 15.27 | 17.27 | 22.14 | 31.54 | 36.59 | 43.87 | 48.44 |
| | 15.78 | 17.18 | 22.62 | 31.61 | 36.73 | 44.14 | 48.48 |
| mean | 15.52 | 17.03 | 22.04 | 29.14 | 36.44 | 43.81 | 49.37 |
| FA-GLA-OH | 6.34 | 14.35 | 19.99 | 23.33 | 31.19 | 43.18 | 49.96 |
| | 4.05 | 8.14 | 16.21 | 23.87 | 33.88 | 43.49 | 48.4 |
| | 4.69 | 9.44 | 14.78 | 24.09 | 33.9 | 43.68 | 49.43 |
| mean | 5.03 | 10.64 | 16.99 | 23.76 | 32.99 | 43.45 | 49.26 |
| H-APA-OH | 13.55 | 14.35 | 23.94 | 24.26 | 28.85 | 44.05 | 48.84 |
| | 8.46 | 14.64 | 24.49 | 24.48 | 29.39 | 41.76 | 49.32 |
| | 7.65 | 14.91 | 25.04 | 28.44 | 29.44 | 43.84 | 49.16 |
| mean | 9.89 | 14.63 | 24.49 | 25.73 | 29.23 | 43.22 | 49.11 |
| H-GLA-OH | 8.37 | 12.4 | 15.53 | 17.58 | 22.67 | 36.63 | 48.16 |
| | 7.42 | 12.53 | 19.03 | 17.94 | 23.33 | 38.42 | 49.91 |
| | 7.12 | 14.66 | 18.34 | 17.53 | 22.93 | 39.4 | 48.18 |
| mean | 7.64 | 13.20 | 17.63 | 17.68 | 22.98 | 38.15 | 48.75 |
| Bn-GLA-OH | 12.92 | 17.74 | 21.14 | 23.01 | 33.30 | 43.67 | 48.53 |
| | 11.17 | 14.86 | 21.54 | 22.71 | 33.45 | 42.91 | 47.02 |
| | 9.65 | 13.38 | 22.01 | 22.90 | 33.40 | 41.17 | 49.55 |
| mean | 11.25 | 15.33 | 21.56 | 22.87 | 33.38 | 42.58 | 48.37 |
| Z-GKA-OH | 8.17 | 12.48 | 14.49 | 21.62 | 23.57 | 42.13 | 49.82 |
| | 9.44 | 14.52 | 16.43 | 21.98 | 23.95 | 42.02 | 49 |
| | 9.44 | 14.82 | 15.03 | 21.52 | 24.36 | 42.51 | 47.7 |
| mean | 9.02 | 13.94 | 15.32 | 21.71 | 23.96 | 42.22 | 48.84 |
| Z-GLA-Nbu | 11.16 | 13.06 | 23.89 | 32.24 | 34.06 | 38.14 | 47.34 |
| | 13.86 | 14.73 | 23.71 | 32.41 | 33.89 | 38.31 | 47 |
| | 14.05 | 14.34 | 24.13 | 32.63 | 34.85 | 36.63 | 48 |
| mean | 13.02 | 14.04 | 23.91 | 32.43 | 34.27 | 37.69 | 47.45 |
| Z-GLA-OH | 1.14 | 6.47 | 11.43 | 14.43 | 21.74 | 32.54 | 49 |
| | 1.44 | 7.66 | 11.9 | 14.26 | 21.93 | 32.61 | 49.4 |
| | 1.55 | 7.49 | 11.46 | 14.37 | 24.44 | 33.41 | 49.5 |
| mean | 1.38 | 7.21 | 11.60 | 14.35 | 22.70 | 32.85 | 49.30 |

### Example 9

### In Vivo Testing of Di- and Tri-Peptides and Derivatives

Table 2 contains in vivo data, showing tumor volume in mm³, in groups of 4 mice with LLC (Lewis Lung Carcinoma). Mice were sacrificed if the tumor volume exceeded 1000 mm³. Some mice were administered with the compounds alone; others were additionally administered with irradiation. Mice were given the compounds, and in some cases also gamma irradition (400 Rad), at days 7, 10, 14, 18 and 21. In combination with irradiation some compounds showed excellent results. The fact that the dipeptide derivative Z-GL-OH performs poorly in vitro as well as in vivo supports the theory that the in vitro results can be extrapolated to in vivo effects.

TABLE 2

| | days after tumor inoculation | | | | |
|---|---|---|---|---|---|
| | 7 | 10 | 14 | 18 | 21 |
| Z-GL-OH | 4.0 | 147.0 | 720.0 | 1687.5 | 1792.0 |
| (comparative) | 10.0 | 171.5 | 660.0 | 1372 | 1352.0 |
| | 8.0 | 192.0 | 936.0 | 840 | |
| | 4.0 | 144.0 | 500.0 | 1176 | |
| mean | 6.5 | 163.63 | 704.0 | 1268.88 | 1572.0 |
| Z-GL-OH | 0.5 | 108.0 | 320.0 | 600 | 1575.0 |
| irradiated | 6.0 | 144.0 | 400.0 | 864 | 1372.0 |
| (comparative) | 6.0 | 90.0 | 112.5 | 840 | 1176.0 |
| | 8.0 | 144.0 | 450.0 | 864 | 1008 |
| mean | 5.13 | 121.5 | 320.63 | 792.0 | 1282.75 |
| PBS (control) | 6.0 | 192.0 | 720.0 | 1575 | |
| | 4.0 | 240.0 | 600.0 | 1568 | |
| | 6.0 | 192.0 | 500.0 | 1274 | |
| | 6.0 | 256.0 | 720.0 | 1008 | |
| | 5.50 | 220.00 | 635.0 | 1356.25 | |
| PBS (control) | 13.5 | 192.0 | 500.0 | 936 | |
| irradiated | 0.5 | 144.0 | 480.0 | 1014 | |
| | 4.0 | 192.0 | 400.0 | 650 | |
| | 6.0 | 144.0 | 600.0 | 600 | |
| mean | 6.00 | 168.00 | 495.00 | 800.00 | |
| FA-GLA-OH | 4.0 | 144.0 | 720.0 | 1176 | |
| | 13.0 | 144.0 | 600.0 | 1687.5 | |
| | 4.0 | 400.0 | 864.0 | 1456 | |
| | 13.0 | 256.0 | 600.0 | 1267.5 | |
| mean | 8.50 | 236.00 | 696.00 | 1396.75 | |
| FA-GLA-OH | 4.0 | 100.0 | 90.0 | 48 | 18.0 |
| irradiated | 0.0 | 90.0 | 120.0 | 48 | 48.0 |
| | 0.5 | 108.0 | 126.0 | 32 | 18.0 |
| | 4.0 | 96.0 | 72.0 | 32 | 12.0 |
| mean | 2.13 | 98.50 | 102.00 | 40.00 | 24.00 |
| H-GLA-OH | 9.0 | 256.0 | 480.0 | 750 | 1792.0 |
| | 0.5 | 126.0 | 864.0 | 1176 | 1280.0 |
| | 0.5 | 126.0 | 480.0 | 1008 | 1890.0 |
| | 18.0 | 320.0 | 864.0 | 1372 | |
| mean | 7.00 | 207.00 | 672.00 | 1076.50 | 1654.00 |
| H-GLA-OH | 13.5 | 62.5 | 256.0 | 108 | 72.0 |
| irradiated | 4.0 | 4.0 | 320.0 | 192 | 108.0 |
| | 0.0 | 60.0 | 320.0 | 192 | 108.0 |
| | 4.0 | 108.0 | 480.0 | 256 | 72.0 |
| mean | 5.38 | 58.63 | 344.00 | 187.00 | 90.00 |
| Bn-GLA-OH | 0.5 | 192.0 | 500.0 | 1575 | 1792.0 |
| | 4.0 | 240.0 | 400.0 | 1372 | 1764.0 |
| | 4.0 | 224.0 | 594.0 | 1008 | |
| | 0.5 | 256.0 | 720.0 | 840 | |
| mean | 2.25 | 228.00 | 553.50 | 1198.75 | 1778.00 |
| Bn-GLA-OH | 4.0 | 144.0 | 144.0 | 48 | 24.0 |
| irradiated | 3.0 | 144.0 | 144.0 | 32 | 4.0 |
| | 8.0 | 171.0 | 171.0 | 4 | 0.5 |
| | 0.5 | 144.0 | 144.0 | 12 | 0.5 |
| mean | 3.88 | 150.75 | 150.75 | 24.00 | 7.25 |
| Z-GLA-OH | 4.0 | 256.0 | 660.0 | 864 | 2048.0 |
| | 0.0 | 192.0 | 864.0 | 1470 | |
| | 6.0 | 9.0 | 720.0 | 1568 | |
| | 13.5 | 144.0 | | | |
| mean | 5.88 | 150.25 | 748 | 1300.67 | |
| Z-GLA-OH | 4.0 | 48.0 | 72.0 | 32 | 13.5 |
| irradiated | 6.0 | 128.0 | 144.0 | 24 | 0.5 |
| | 0.0 | 40.0 | 72.0 | 13.5 | 13.5 |
| | 6.0 | 40.0 | 48.0 | 32 | 0.5 |
| mean | 4.00 | 64.00 | 84.00 | 25.38 | 7.00 |

### Example 10

### Further In Vivo Testing of Z-GLA-OH

Table 3 contains further in vivo data, showing tumor volume in mm³, in groups of 7-8 mice, according to the EL-4 tumor model described above. 1.000.000 EL-4 lymphoma cells were inoculated subcutaneously at day 0. No palpable tumors were observed until day 22. At each treatment (twice weekly) mice with palpable tumors were given 400 Rads irradiation alone, or in combination with 14 micro-l 50 mM solution of Z-GLA-OH. Mice with no palpable tumors were not treated, i.e. in mice with rejected tumors, treatment was terminated and the mice were kept under observation. Table 3 shows excellent results, namely complete rejection of established tumors, not just arrest of tumor growth, decreased volume, or a delay of tumor growth.

All mice were 400 Rad (1 Gy=100 Rad) gamma-irradiated at day 0, a standard procedure to improve tumor acceptance. The compound was inoculated intraperitoneally, whereas tumors were always inoculated subcutaneously.

TABLE 3

| | | | irradiation (*) | | |
|---|---|---|---|---|---|
| | | | no add | irradiation | irradiation |
| | | | Z-GLA-OH (#) | | |
| | | | no add | no add | Z-GLA-OH |
| Day | # | 22 | 0.50 | 4.00 | 0.50 |
| | | | 0.50 | 0.50 | 0.50 |
| | | | 13.50 | 0.50 | 4.00 |
| | | | 108.00 | 4.00 | 6.00 |
| | | | 4.00 | 13.50 | 0.50 |
| | | | 4.00 | 0.50 | 0.50 |
| | | | 0.50 | 4.00 | 4.00 |
| | | | 0.50 | | 0.50 |
| mean | | | 16.44 | 3.86 | 2.06 |
| | # and * | 26 | 72.00 | 75.00 | 108.00 |
| | | | 256.00 | 108.00 | 126.00 |
| | | | 75.00 | 13.50 | 108.00 |
| | | | 108.00 | 24.00 | 75.00 |
| | | | 108.00 | 13.50 | 50.00 |
| | | | 48.00 | 90.00 | 40.00 |
| | | | 60.00 | 62.50 | 62.50 |
| | | | 90.00 | | 108.00 |
| mean | | | 102.13 | 55.22 | 84.69 |
| | # and * | 30 | 500.00 | 192.00 | 108.00 |
| | | | 192.00 | 64.00 | 13.50 |
| | | | 192.00 | 192.00 | 18.00 |
| | | | 256.00 | 144.00 | 24.00 |
| | | | 500.00 | 192.00 | 32.00 |
| | | | 400.00 | 256.00 | 48.00 |
| | | | 256.00 | 432.00 | 108.00 |
| | | | 320.00 | | 48.00 |
| mean | | | 327.00 | 210.28 | 49.94 |
| | # and * | 34 | 864.00 | 90.00 | 10.00 |
| | | | 256.00 | 144.00 | 0.50 |
| | | | 400.00 | 400.00 | 62.50 |
| | | | 500.00 | 256.00 | 24.00 |
| | | | 480.00 | 320.00 | 13.50 |
| | | | 400.00 | 400.00 | 18.00 |
| | | | 600.00 | 240.00 | 24.00 |
| | | | 400.00 | | 13.50 |
| mean | | | 487.50 | 264.28 | 20.75 |
| | # and * | 37 | 720.00 | 500.00 | 8.00 |
| | | | 320.00 | 400.00 | 18.00 |
| | | | 320.00 | 550.00 | 12.00 |
| | | | 600.00 | 600.00 | 6.00 |
| | | | 600.00 | 320.00 | 27.00 |
| | | | 320.00 | 320.00 | 6.00 |
| | | | 576.00 | 396.00 | 24.00 |
| | | | 720.00 | | 0.50 |
| mean | | | 522.00 | 440.85 | 12.69 |
| | # and * | 41 | 1170.00 | 480.00 | 0.50 |
| | | | 840.00 | 480.00 | 4.00 |
| | | | 1092.00 | 480.00 | 4.00 |
| | | | 900.00 | 600.00 | 13.50 |
| | | | 720.00 | 780.00 | 4.00 |
| | | | 1176.00 | 800.00 | 0.50 |
| | | | 1008.00 | 480.00 | 4.00 |
| | | | 910.00 | | 0.50 |
| mean | | | 977.00 | 585.72 | 3.88 |
| | # and * | 44 | 1800.00 | 1008.00 | 0.50 |
| | | | 1920.00 | 720.00 | 0.10 |
| | | | 2304.00 | 726.00 | 0.50 |
| | | | 2304.00 | 720.00 | 2.00 |
| | | | 2160.00 | 1008.00 | 0.50 |
| | | | 1764.00 | 1792.00 | 1.00 |
| | | | 1920.00 | 1008.00 | 0.50 |
| | | | 1792.00 | | 4.00 |
| mean | | | 1995.50 | 997.43 | 1.14 |
| | # and * | 48 | sacrificed | 1344.00 | 0.10 |
| | | | | 1920.00 | 0.50 |
| | | | | 1575.00 | 0.50 |
| | | | | 2048.00 | 0.10 |
| | | | | 2048.00 | 0.10 |
| | | | | 2304.00 | 0.10 |
| | | | | | 0.00 |
| | | | | | 0.00 |
| mean | | | | 1873.16 | 0.18 |
| | # and * | 55 | | sacrificed | 0.00 |
| | | | | | 0.00 |
| | | | | | 0.00 |
| | | | | | 0.00 |
| | | | | | 0.10 |
| | | | | | 0.00 |
| | | | | | 0.00 |
| | | | | | 0.50 |
| mean | | | | | 0.07 |
| | # and * | 62 | | | 0.10 |
| | | | | | 0.00 |
| | | | | | 0.00 |
| | | | | | 0.00 |
| | | | | | 0.00 |
| | | | | | 0.00 |
| | | | | | 0.00 |
| | | | | | 0.00 |
| mean | | | | | 0.01 |
| | # and * | 65 | | | 0.10 |
| | | | | | 0.00 |
| | | | | | 0.00 |
| | | | | | 0.00 |
| | | | | | 0.00 |
| | | | | | 0.00 |
| | | | | | 0.00 |
| | | | | | 0.00 |
| mean | | | | | 0.01 |
| | # and * | 72 | | | 0.10 |
| | | | | | 0.00 |
| | | | | | 0.00 |
| | | | | | 0.00 |
| | | | | | 0.00 |
| | | | | | 0.00 |
| | | | | | 0.00 |
| | | | | | 0.00 |
| mean | | | | | 0.01 |
| | | 78 | | | 0.00 |
| | | | | | 0.00 |
| | | | | | 0.00 |
| | | | | | 0.00 |
| | | | | | 0.00 |
| | | | | | 0.00 |
| | | | | | 0.00 |
| | | | | | 0.00 |
| mean | | | | | 0.00 |

### Example 11 and FIG. 8

We tested GPG-NH₂ and Z-GPG-NH₂ in the same manner as Z-GLA-OH. These were injected twice weekly at 13.8 mg/kg in tumor bearing mice, and compared to Z-GLA-OH for their ability to mediate sensitization to gamma-irradiation in vivo. We found that both GPG-NH₂ and Z-GPG-NH₂ mediated complete regression of established EL-4 tumors following gamma-irradiation.

### Example 12 and FIG. 9

### TPP II is Required for Mre11 Foci Formation

As shown in FIG. 1C and discussed under Example 1, TPPII is rapidly translocated into the nucleus of gamma-irradiated cells. The results of further immunocytochemical experiments are shown in FIG. 9. TPPII does not appear to form foci, which would have instead shown a dotted appearance (FIG. 9, shown for cells with inhibited TPPII expression, LLC, ALC and YAC-1). This failure of cells with inhibited TPP II expression to assemble Mre11 foci upon gamma-irradiation exposure provides further support for the use of TPP II inhibitors in the present invention.

1. A method of enhancing the efficacy of gamma-irradiation cancer therapy or increasing the in vivo gamma-irradiation susceptibility of tumour cells comprising administering to a patient in need thereof a therapeutically effective amount of a TPP II inhibitor compound.

2. A method as claimed in claim 1, wherein said compound is selected from formula (i) or is a pharmaceutically acceptable salt thereof:
R^{N1}R^{N2}N A¹-A²-A³-CO—R^{C1} (i)
wherein A¹, A² and A³ are amino acid residues having the following definitions according to the standard one-letter amino acid abbreviations or names:
A¹ is G, A, V, L, I, P, 2-aminobutyric acid, norvaline or tert-butyl glycine,
A² is G, A, V, L, I, P, F, W, C, S, K, R, 2-aminobutyric acid, norvaline, norleucine, tert-butyl alanine, alpha-methyl leucine, 4,5-dehydro-leucine, allo-isoleucine, alpha-methyl valine, tert-butyl glycine, 2-allylglycine, ornithine or alpha, gamma-diaminobutyric acid,
A³ is G, A, V, L, I, P, F, W, D, E, Y, 2-aminobutyric acid, norvaline or tert-butyl glycine,
R^{N1} and R^{N2} are each attached to the N terminus of the peptide, are the same or different, and are each independently
R^{N3},
(linker1)-R^{N3},
CO-(linker1)-R^{N3},
CO—O-(linker1)-R^{N3},
CO—N-((linker1)-R^{N3}))R^{N4} or
SO₂—(linker1)-R^{N3},
(linker 1) may be a single bond, or CH₂, CH₂CH₂, CH₂CH₂CH₂, CH₂CH₂CH₂CH₂ or CH═CH,
R^{N3} and R^{N4} are the same or different and are hydrogen or any of the following optionally substituted groups:
saturated or unsaturated, branched or unbranched C₁₋₆ alkyl;
saturated or unsaturated, branched or unbranched C₃₋₁₂ cycloalkyl;
benzyl;
phenyl;
naphthyl;
mono- or bicyclic C₁₋₁₀ heteroaryl; or
non-aromatic C₁₋₁₀ heterocyclyl;
wherein there may be zero, one or two same or different optional substituents on R^{N3} and/or R^{N4} which may be:
hydroxy-;
thio-:
amino-;
carboxylic acid;
saturated or unsaturated, branched or unbranched C₁₋₆ alkyloxy;
saturated or unsaturated, branched or unbranched C₃₋₁₂ cycloalkyl;
N—, O—, or S— acetyl;
carboxylic acid saturated or unsaturated, branched or unbranched C₁₋₆ alkyl ester;
carboxylic acid saturated or unsaturated, branched or unbranched C₃₋₁₂ cycloalkyl ester
phenyl;
mono- or bicyclic C₁₋₁₀ heteroaryl;
non-aromatic C₁₋₁₀ heterocyclyl; or
halogen; and
R^{C1} is attached to the C terminus of the tripeptide, and is:
O—R^{c2},
O-(linker2)-R^{C2},
N((linker2)R^{C2})R^{C3}, or
N(linker2)R^{C2}—NR^{C3}R^{C4},
wherein (linker2) may be a single bond, or C₁₋₆ alkyl or C₂₋₄ alkenyl, and
R^{C2}, R^{C3} and R^{C4} are the same or different, and are hydrogen or any of the following optionally substituted groups:
saturated or unsaturated, branched or unbranched C₁₋₆ alkyl;
saturated or unsaturated, branched or unbranched C₃₋₁₂ cycloalkyl;
benzyl;
phenyl;
naphthyl;
mono- or bicyclic C₁₋₁₀ heteroaryl; or
non-aromatic C₁₋₁₀ heterocyclyl;
wherein there may be zero, one or two same or different optional substituents on each of R^{C2} and/or R^{C3} and/or R^{C4} which may be one or more of:
hydroxy-;
thio-:
amino-;
carboxylic acid;
saturated or unsaturated, branched or unbranched C₁₋₆ alkyloxy;
saturated or unsaturated, branched or unbranched C₃₋₁₂ cycloalkyl;
N—, O—, or S— acetyl;
carboxylic acid saturated or unsaturated, branched or unbranched C₁₋₆ alkyl ester;
carboxylic acid saturated or unsaturated, branched or unbranched C₃₋₁₂ cycloalkyl ester
phenyl;
halogen;
mono- or bicyclic C₁₋₁₀ heteroaryl; or
non-aromatic C₁₋₁₀ heterocyclyl.

3. A method as claimed in claim 2 wherein said compound of formula (i) is such that:
R^{N1} is hydrogen,
R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

4. A method as claimed in claim 3, wherein said compound of formula (i) is such that:
A¹ is G, A or 2-aminobutyric acid,
A² is L, I, norleucine, V, norvaline, tert-butyl alanine, 4,5-dehydro-leucine, allo-isoleucine, 2-allylglycine, P, 2-aminobutyric acid, alpha-methyl leucine, alpha-methyl valine or tert-butyl glycine,
A³ is G, A, V, P, 2-aminobutyric acid or norvaline,
R^{N1} is H,
R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

5. A method as claimed in claim 4, wherein said compound of formula (i) is such that:
A¹ is G, A or 2-aminobutyric acid,
A² is L, I, norleucine, V, norvaline, tert-butyl alanine, 4,5-dehydro-leucine, allo-isoleucine or 2-allylglycine,
A³ is G, A, V, P, 2-aminobutyric acid or norvaline,
R^{N1} is H,
R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

6. A method as claimed in claim 5 wherein said compound of formula (i) is such that:
A¹ is G or A,
A² is L, I, or norleucine,
A³ is G or A,
R^{N1} is hydrogen,
R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

7. A method as claimed in any of claims 2 to 6 wherein
R^{N1} is hydrogen,
R^{N2} is hydrogen, C(═O)—OCH₂Ph or C(═O)—CH═CH-(2-furyl), and
R^{C1} is OH, O—C₁₋₆ alkyl, or NH—C₁₋₆ alkyl.

8. A method as claimed in claim 7 wherein said compound of formula (i) is Z-GLA-OH, Bn-GLA-OH, FA-GLA-OH or H-GLA-OH.

9. A method as claimed in claim 8 wherein said compound of formula (i) is Z-GLA-OH.

10. A method as claimed in claim 2 wherein A¹ is G, A or 2-aminobutyric acid.

11. A method as claimed in claim 10 wherein A¹ is G or A.

12. A method as claimed in claim 2, wherein A² is L, I, norleucine, V, norvaline, tert-butyl alanine, 4,5-dehydro-leucine, allo-isoleucine, 2-allylglycine, P, K, 2-aminobutyric acid, alpha-methyl leucine, alpha-methyl valine or tert-butyl glycine.

13. A method as claimed in claim 12 wherein A² is L, I, norleucine, V, norvaline, tert-butyl alanine, 4,5-dehydro-leucine, allo-isoleucine, 2-allylglycine, P or K.

14. A method as claimed in claim 13 wherein A² is L, I, norleucine, P or K.

15. A method as claimed in claim 14 wherein A² is L or P.

16. A method as claimed in claim 15 wherein A² is P.

17. A method as claimed in claim 2 wherein A³ is G, A, V, P, 2-aminobutyric acid or norvaline.

18. A method as claimed in claim 17 wherein A³ is G or A.

19. A method as claimed in claim 2 wherein R^{N1} is hydrogen.

20. A method as claimed in claim 2 wherein R^{N2} is
R^{N3},
(linker1)-R^{N3},
CO-(linker1)-R^{N3}, or
CO—O-(linker1)-R^{N3},
wherein
(linker1) may be a single bond, or CH₂, CH₂CH₂, CH₂CH₂CH₂, CH₂CH₂CH₂CH₂ or CH═CH, and
R^{N3} is hydrogen or any of the following unsubstituted groups:
saturated or unsaturated, branched or unbranched C₁₋₄ alkyl;
benzyl;
phenyl; or
monocyclic heteroaryl.

21. A method as claimed in claim 20 wherein R^{N2} is hydrogen, benzyloxycarbonyl, benzyl, benzoyl, tert-butyloxycarbonyl, 9-fluorenylmeth-oxycarbonyl or FA.

22. A method as claimed in claim 21 wherein R^{N2} is hydrogen, benzyloxycarbonyl or FA.

23. A method as claimed in claim 2 wherein R^{C1} is:
O—R^{C2},
O-(linker2)-R^{C2}, or
NH-(linker2)R^{C2}
wherein
(linker2) may be a single bond, C₁₋₆ alkyl or C₂₋₄ alkenyl, and
R^{C2} is hydrogen or any of the following unsubstituted groups:
saturated or unsaturated, branched or unbranched C₁₋₅ alkyl;
benzyl;
phenyl; or
monocyclic C₁₋₁₀ heteroaryl.

24. A method as claimed in claim 23 wherein R^{C1} is OH, O—C₁₋₆ alkyl, O—C₁₋₆ alkyl-phenyl, NH₂, NH—C₁₋₆ alkyl, or NH—C₁₋₆ alkyl-phenyl.

25. A method as claimed in claim 24 wherein R^{C1} is OH, O—C₁₋₆ alkyl, NH₂, or NH—C₁₋₆ alkyl.

26. A method as claimed in claim 25 wherein R^{C1} is OH or NH₂.

27. A method as claimed in claim 26 wherein R^{C1} is NH₂.

28. A method as claimed in claim 2 wherein said compound is GPG-NH₂, Z-GPG-NH₂, Bn-GPG-NH₂, FA-GPG-NH₂, GPG-OH, Z-GPG-OH, Bn-GPG-OH, or FA-GPG-OH.

29. A method as claimed in claim 28 wherein said compound is GPG-NH₂.

30. A method as claimed in claim 2 wherein said compound is ALG-NH₂, Z-ALG-NH₂, Bn-ALG-NH₂, FA-ALG-NH₂, ALG-OH, Z-ALG-OH, Bn-ALG-OH, or FA-ALG-OH.

31. A method as claimed in claim 30 wherein said compound is ALG-NH₂.

32-36. (canceled)

37. A method for identifying a compound suitable for enhancing the efficacy of gamma-irradiation cancer therapy or increasing the in vivo gamma-irradiation susceptibility of tumour cells comprising contacting TPP II with a compound to be screened, and identifying whether the compound inhibits the activity of TPP II.

38. A pharmaceutical composition comprising a compound of formula (i) as defined in claim 2 and a pharmaceutically acceptable diluent or carrier, with the proviso that said compound is not selected from any of the following:
(a) GPE-OH;
(b) a compound of the formula 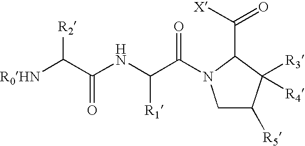
Wherein X′ represents OH, (C₁₋₅)alkoxy, NH₂, NH—C₁₋₅-alkyl, N(C₁₋₅ alkyl)₂;
R₁′ is a residue derived from any of the amino acids Phe, Tyr, Trp, Pro, each of which may optionally be substituted by a (C₁₋₅)alkoxy group, a (C₁₋₅)alkyl group or a halogen atom, and Ala, Val, Leu, or Ile;
R₂′ is a residue which is derived from any of the amino acids Gly, Ala, Ile, Val, Ser, Thr, His, Arg, Lys, Pro, Glu, Gln, pGlu, Asp, Leu and Asn;
R₃′ and R₄′ independently represent H, OH, (C₁₋₅)alkyl, or (C₁₋₅)alkoxy, provided that R₃′ and R₄′ are not both OH or (C₁₋₅)alkoxy;
R₅′ represents H, OH, (C₁₋₅)alkyl or (C₁₋₅)alkoxy;
and wherein R₀′ represents a group of the formula 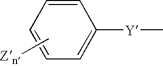
wherein Y′ represents —CO—, —CH₂CO—, —CH₂CH₂CO—, —CH₂CH₂CH₂CO—, —CH═CH—CO or —OCH₂CO—, and wherein Z′ represents a halogen atom, a trifluormethyl group, (C₁₋₄) alkoxy group, (C₁₋₄) alkyl group; or wherein two neighbouring substituents may form a (C₁₋₃) alkylene-dioxy group; and wherein n′ is 0 or an integer of from 1 to 5;
(c) X″-PG-NH₂, wherein X″ is an amino acid residue;
(d) PGP-OH;
(e) any of the following compounds
GPG-NH₂
GKG-NH₂
CQG-NH₂
RQG-NH₂
KQG-NH₂
ALG-NH₂
GVG-NH₂
VGG-NH₂
ASG-NH₂
SLG-NH₂
SPT-NH₂;
(f) any of the following compounds
AIG-NH₂
GFG-NH₂
GWG-NH₂
FLG-NH₂
GYG-NH₂
APG-NH₂
GLG-NH₂
tBu-GPG-NH₂
(g) LAP-OH
(h) a compound comprising the sequence GPX′″ wherein X′″ is an amino acid
(i) IVY-OH
(j) GFE-OH
(k) any of the following compounds
VPP-OH
IPP-OH
(l) PRG-NH₂; and
(m) any of the following compounds
PLG-NH₂
PAG-NH₂
GPG-OH
PG-ARG-NH₂
GPA-NH₂
GGG-NH₂
LKA-NH₂
ILK-NH₂
GPQ—NH₂
GHK-NH₂
ACQ—NH₂
ARV-NH₂
KAR-NH₂
HKA-NH₂
GAT-NH₂
KAL-NH₂
PGR-NH₂
GhydPG-NH₂
tBu-GLG-NH₂
metALG-NH₂ and
LNF-NH₂.

39-42. (canceled)

43. A pharmaceutical composition comprising a compound as defined in claim 6 and a pharmaceutically acceptable diluent or carrier.

44-45. (canceled)
